(19) United States
(12) Patent Application Publication
(10) Pub. No.: US 2014/0023984 A1
(43) Pub. Date: Jan. 23, 2014

(54) DENTAL CHARTING SYSTEM

(71) Applicants: Paul Deane Weatherly, Auckland (NZ); Shane Allan Blackett, Auckland (NZ); Paul David Harris, Lower Hutt (NZ); Russell Petherick, Lower Hutt (NZ)

(72) Inventors: Paul Deane Weatherly, Auckland (NZ); Shane Allan Blackett, Auckland (NZ); Paul David Harris, Lower Hutt (NZ); Russell Petherick, Lower Hutt (NZ)

(21) Appl. No.: 13/996,810

(22) PCT Filed: Dec. 22, 2011
(86) PCT No.: PCT/NZ11/00272

§ 371 Date: Sep. 3, 2013

(30) Foreign Application Priority Data
Dec. 22, 2010 (NZ) 590155

(51) Int. Cl. A61B 1/247; G06F 3/01

(52) U.S. Cl. CPC A61B 1/247; G06F 3/017
USPC 433/31; 345/156

(57) ABSTRACT

A dental charting system having a control device configured to be held by a user or which is mounted to another device held by a user and the control device including one or more movement sensors for sensing movement of the control device in space and generating representative movement signals. A gesture processing module is provided to receive and process the movement signals to detect the occurrence of a gesture event from a set of predetermined gesture events, and which generates representative gesture control signals. A dental charting application program is also provided and runs on a host device and which provides a graphic user interface on a display associated with the host device for interacting with the application program, the application program being configured to receive and process the gesture control signals to enable interaction with the graphic user interface by the user via hand gesture movements of the control device.

### FIELD OF THE INVENTION

The present invention relates to a dental charting system for recording, viewing, maintaining, and updating electronic patient records, such as dental and periodontal charts.

### BACKGROUND TO THE INVENTION

The use of computers and associated software for maintaining electronic patient records are now commonplace in dental examination and treatment rooms. While real-time maintenance of electronic patient records during treatment increases efficiency in most dental practices, the use of computing devices in clinical environments raises challenges in itself. First, the use of a computing device during treatment raises infection and contamination issues. For example, each time the dentist, hygienist or other operator uses the keyboard or mouse there is a risk of transfer of bacteria and viruses between the patients and anyone operating the computers. This issue is often dealt with by the dentist changing their surgical gloves after each interaction with the computer, although this is costly and inefficient. Second, the use of computers in the treatment room creates inefficiencies as the dentist is constantly swapping between operating the computer input devices, such as the keyboard and mouse, to update clinical information, and then back to using the sterile dental instruments and treating the patient.

It is an object of the present invention to provide an improved dental charting system, or to at least provide the public with a useful choice.

### SUMMARY OF THE INVENTION

In a first aspect, the present invention broadly consists in a dental charting system, comprising:
a control device configured to be held by a user or which is mounted to another device held by a user and the control device comprising one or more movement sensors for sensing movement of the control device in space and generating representative movement signals;
a gesture processing module configured to receive and process the movement signals to detect the occurrence of a gesture event from a set of predetermined gesture events, and which generates representative gesture control signals; and
a dental charting application program running on a host device and which provides a graphic user interface on a display associated with the host device for interacting with the application program, the application program being configured to receive and process the gesture control signals to enable interaction with the graphic user interface by the user via hand gesture movements of the control device.

In one form, the host device may be in the form of a computer system, such as a personal computer whether in the form of a desktop, laptop or other portable computing device or system and which has an associated visual display for displaying a GUI to enable a user to interact with the computer system.

In another form, the host device may be in the form of a hardware device comprising a processor, memory and onboard display and which is in direct or indirect signal communication with the control device. By way of example only, the hardware device may be purpose-built or configured for the dental charting system. Preferably, the hardware device is configured to store patient data representing the user's interaction with the dental charting application program during a session. More preferably, the host device may transfer the patient data to another main or central data system. In one form, the main or central data system may be a computer system running an electronic patient records system.

In one form, the control device is a portable handheld device. In one embodiment, the control device may be in the form of a dental instrument. In another embodiment, the control device may be in the form of an elongate control wand.

In another form, the control device may be fixedly mounted or releasably mounted to a dental instrument.

The following features are described for an embodiment in which the control device is in the form of or attached to a dental instrument, but the features equally apply to other forms of the control device, such as the control wand form or otherwise.

In one form, the dental instrument is in direct signal communication with the host device. In another form, the dental instrument is in signal communication with the host device via an intermediate interface device. The signal communication medium(s) between the dental instrument, intermediate interface device, and host device, as the case may be, may be wired, wireless, or a combination of these communication mediums.

In a first embodiment, the gesture processing module is provided in the dental instrument such that the dental instrument generates and sends the gesture control signals either directly to the host device or indirectly to the host device via the intermediate interface device.

In a second embodiment, the gesture processing module is provided in the host device such that the host device generates the gesture control signals based on the movement signals received directly from the dental instrument or indirectly from the dental instrument via an intermediate interface device. In one form, the host device may be a personal computer and the gesture processing module may be provided in the form of a device driver running on the operating system for translating the movement signals from the dental instrument into gesture control signals for sending to the dental charting application program.

Preferably, the communication mediums between the dental instrument, intermediate interface device, and host device, as the case may be, are bidirectional. More preferably, the application program may send status signals to the dental instrument that are indicative of application program actions or events, or the host device status, for example. Any of the dental instrument, intermediate interface device, and host device may be provided with audible, visual or tactile feedback devices, such as buzzers, LEDS, displays (e.g. LCD), vibration devices or the like.

In a third embodiment, the gesture processing module may be provided in the intermediate interface device such that the intermediate interface device receives and processes the movement signals from the dental instrument to generate the gesture control signals for sending to the host device.

In a fourth embodiment, the functionality and processing of the gesture processing module may be spread or distributed across two or more of the dental instrument, intermediate interface device, and the host device, as the case may be.

Preferably, the dental instrument has a predefined reference axis. In one form, the dental instrument is substantially elongate and the reference axis is aligned or parallel with the longitudinal axis of the instrument.

In one embodiment, the movement sensor(s) generate movement signals that represent movement of the dental instrument with respect to a local reference frame of the instrument. In one form, the movement sensor(s) may be inertial sensor(s). For example, the inertial sensor may comprise a gyroscope sensor mounted to or within the dental instrument that is configured to sense rotation of one or more reference axes of the dental instrument with reference to one or more instrument (control device) reference planes defined by the local reference frame and generate representative instrument rotation signals.

In another embodiment, the movement sensor(s) generate movement signals that represent rotation of the reference axis with reference to one or more global reference planes oriented with respect to gravity. By way of example, the global reference plane(s) may comprise a horizontal plane with reference to gravity, a vertical plane with reference to gravity, or both. Preferably, the horizontal and/or vertical planes are aligned with the reference axis of the dental instrument.

In one form, the movement sensors may be inertial sensors. For example, the inertial sensors may comprise: an accelerometer sensor mounted to or within the dental instrument that is configured to sense the orientation of the reference axis of the dental instrument with respect to gravity and generate representative orientation signals; and a gyroscope sensor mounted to or within the dental instrument that is configured to sense rotation of the reference axis of the dental instrument with reference to one or more instrument reference planes and generate representative instrument rotation signals.

In one form, the accelerometer sensor may be in the form of a three-axis accelerometer which may be configured such that at least one of the sensor axes is co-aligned or parallel with the reference axis of the dental instrument.

In one form, the gyroscope sensor may be in the form of a two-axis gyroscope that is configured to sense rotation of the reference axis with reference to two perpendicular instrument reference planes. Preferably, either or both of the instrument reference planes may have an orientation that is co-aligned or parallel to the reference axis of the dental instrument. By co-aligned, it is meant that the reference axis lies in the instrument reference plane. More preferably, the perpendicular instrument reference planes are both co-aligned with the reference axis of the dental instrument such that the reference axis lies along the intersection of the two planes.

In other forms, the accelerometer and gyroscope sensors may be arbitrarily mounted relative to the reference axis of the dental instrument and each other, and calibration of the sensor signals relative to each to other and with the reference axis may be performed during an initial device configuration or setup. By way of example, a calibration module may perform the sensor calibration to determine the relationship between the sensor signals. The calibration module may be provided in any suitable part of the system.

In one form, the gesture processing module comprises a movement processing sub-module and a gesture detection sub-module.

Preferably, the movement processing sub-module is configured to receive and process the raw accelerometer and gyroscope signals to generate global rotation angle signals representing the rotation of the reference axis of the dental instrument with reference to one or more global reference planes oriented with respect to gravity. More preferably, the movement processing sub-module is configured to process the accelerometer orientation signals to extract the pitch and roll of the reference axis of the dental instrument with respect to gravity and generate representative pitch and roll signals; and is further configured to convert the instrument rotation signals from the gyroscope into global rotation signals representing the rotation of the reference axis of the dental instrument with respect to the one or more global reference planes based on the pitch and roll signals. In effect, the movement processing sub-module is configured to convert or adjust the gyroscope instrument rotation signals into a reference frame oriented with respect to gravity.

In one form, the global rotation signals may comprise a global yaw signal representing the rotation of the reference axis of the dental instrument in the horizontal plane with respect to gravity and a global pitch signal representing the rotation of the reference axis in the vertical plane with respect to gravity.

Preferably, the instrument rotation angles and orientation-adjusted global rotation signals represent change in rotation (angular velocity).

Preferably, the gesture detection sub-module is configured to receive the global rotation signals from the movement processing sub-module, process the global rotation signals to detect the occurrence of gesture events, and generate representative gesture control signals representing any detected gesture events for sending to the host device and/or application program.

Preferably, the gesture detection sub-module comprises a set of predetermined gesture events for detecting, each gesture event in the set being defined by a movement sequence based on the change of one or more of the global rotation signals relative to predefined signal threshold(s) and/or timing profile(s). More preferably, each gesture event is defined by a signal fluctuation profile in regard to one or more of the movement signals. By way of example, the fluctuation profile may be defined by signal magnitude and/or polarity against time.

In one embodiment, the set of gesture events may comprise one or more directional gesture events, such as, but not limited to: left gesture, right gesture, up gesture, and down gesture. Preferably, each of the directional gestures is defined by a movement sequence comprising an initial trigger movement causing a change in rotation in a respective (e.g. left for left gesture) direction of a magnitude that exceeds a trigger threshold, followed by a return movement causing a subsequent change in rotation in the opposite direction of a magnitude exceeding a return threshold. More preferably, the movement sequence requires the return movement to occur within a predefined timeout period after detection of the initial trigger movement to complete the movement sequence such that a direction or gesture event is registered as occurring.

Preferably, the horizontal directional or gesture events (left gesture and right gesture) are defined by movement sequences defined by changes in the global yaw signal. More preferably, a horizontal directional gesture event is detected if the global yaw signal exceeds a trigger threshold in either direction (representing a trigger movement in either left or right directions) followed by the global yaw signal exceeding a return threshold in the opposite direction (representing a return movement in the opposite direction). By way of example, the polarity of the global yaw signal represents the direction of movement. For example, the positive global yaw signal may represent a rotation in the left direction, and a negative global yaw signal may represent a rotation in the right direction, or vice versa if desired.

Preferably, the vertical directional gesture events (up gesture and down gesture) are defined by movement sequences defined by changes in the global pitch signal. More preferably, a vertical directional gesture event is detected if the global pitch signal exceeds a trigger threshold in either direction (representing a trigger movement in either up or down directions) followed by the global pitch signal exceeding a return threshold in the opposite direction (representing a return movement in the opposite direction). By way of example, the polarity of the global pitch signal represents the direction of movement. For example, the positive global pitch signal represents a rotation in a downward direction, and a negative global yaw signal represents a rotation in an upward direction, or vice versa if desired.

Preferably, the gesture detection sub-module is configured to only detect gesture events if the movement of the dental instrument is initially in a steady state. More preferably, the movement processing sub-module is configured to determine whether a steady state exists based on the magnitude of the global rotation signals relative to a steady state threshold. For example, a steady state is met when the absolute magnitude of the global rotation signals are below a steady state threshold level.

In one embodiment, the dental instrument further comprises one or more physical or virtual control switches that are operable or triggerable by the user to generate switch signals representing operation of the one or more switches. The raw switch signals or alternatively processed switch signals are received in the form of action control signals by the application program to enable further interaction with the application program by the user. Preferably, the action control signals represent the occurrence of action events from a set of predetermined action events that enable the user to interact with the application program via the GUI in combination with navigating via the gesture control signals. By way of example, each action event may be defined by a predefined switch operation or sequence of switch activation and/or deactivation of one or more switches and may be dependent on timing of the switch activation and/or deactivation. For example, a double tap action event may be defined by a double activation of the switch within a predetermined time period. Alternatively, another action event may be dependent on the activation of a switch for a predetermined time period (e.g. short press or long press). Further alternative activation events may be defined by the activation of various combinations of two or more switches simultaneously, or ordered sequence of switch activation of two or more switches.

In another form, the gesture processing module is configured to receive the instrument rotation signals from the gyroscope sensor, process those signals to detect the occurrence of gesture events, and generate representative gesture control signals representing any detected gesture events for sending to the host device and/or application program. In this form, the gestures are directly detected based on relative movements of the dental instrument, and this embodiment does not require an accelerometer sensor in the dental instrument to provide a reference to gravity. In this embodiment, the gestures are defined with respect to the dental instrument local reference frame, not with respect to gravity. By way of example, the direction of gestures may be defined by movements relative to a fixed axes, references or parts (e.g. mirror) of the dental instrument.

In one form, the dental instrument is provided with one or more physical switches, operation of the physical switches generating a respective switch signal. The physical switches may be in any suitable form for pressing by the finger of a user, including, but not limited to: mechanical switches, such as microswitches, or touch switches, such as capacitive switches.

In an alternative form, the dental instrument may be provided with one or more virtual switches that are activated or triggered via movement of the dental instrument as detected by one or more of the movement sensors, such as inertial sensors. By way of example, the virtual switch may be in the form of a tap detection module that is configured to detect ‘single taps’ or ‘double taps’ and generates representative switch signals. In one form, the tap detection module may be integrated with the accelerometer sensor. In an alternative form, the tap detection module may be provided in the dental instrument and configured to receive and process the raw accelerometer from accelerometer sensor signals for generating the switch signals representing detected single taps or double taps.

In one embodiment, the system further comprises an action processing module that is configured to receive and process the switch signals from the physical and/or virtual switches of the dental instrument to detect the occurrence of action events from a set of predetermined action events, and which generates representative action control signals for the application program. The action processing module may be provided in the dental instrument, in the host device, or in the intermediate interface device, or its functionality and processing may be spread or distributed across two or more of the devices. In one embodiment where the host device is a personal computer, the gesture processing module and action processing module may be provided in the form of a device driver that receives the movement signals and switch signals from the dental instrument and generates the corresponding gesture control signals and action control signals for sending to the application program for processing to enable the user to interact with the program via the GUI with a combination of gesture movements of the dental instrument and/or switch activation.

Preferably, the portable handheld dental instrument comprises an elongate handle portion and a tool portion extending from an end of the handle portion, and the handle portion comprising a substantially elongate main body within which the movement sensors are mounted and an outer casing that is configured to substantially surround the entire or at least a substantial portion of the main body.

Preferably, the outer casing is in the form of an elongate sleeve that fits over a substantial portion of the main body. For example, the entire main body or a substantial portion of the main body may be slidably received and retained within the hollow sleeve. The sleeve may be formed from a substantially rigid material, and may be for example molded from plastic.

Preferably, the dental instrument further comprises one or more touch switches mounted to the surface of the main body and wherein the main body and outer casing are configured such that there is an air gap between the touch switches and the inner surface of the outer casing in the vicinity of the touch switches. In one form, the main body is provided with a recessed surface within which the touch switches are mounted so as to provide the air gap between the touch switches and the outer casing. In another form, the outer casing is provided with a recessed surface or reduced thickness in a region aligned with the touch switches of the main body so as to provide the air gap between the touch switches and the outer casing.

Preferably, the outer casing of the dental instrument is resiliently deformable in a region aligned with the touch switches of the main body.

The tool portion may be integrally formed with the handle portion or alternatively may be removably detachable from the handle portion.

In use, the outer casing is removable and may be disposable and replaceable for each patient, or alternatively may be removed from the main body of the dental instrument for sterilization between each patient.

The outer casing may be configured to have a snap-fit, or friction-fit engagement with the main body or may be removably mounted to the main body via an operable latching or locking mechanism.

The tool portion may be a mirror, probe or any other suitable dental tool. In one embodiment, the tool portion is a snap-in or screw-thread mounted mirror that is releaseably mountable to an end of the handle portion.

In one embodiment, the GUI comprises a set of display GUIs that represent the stage or progression of the user through the workflow of the application program.

Preferably, each display GUI may be icon-based. More preferably, each display GUI may comprise an arrangement or configuration of icons that may be selected and activated to progress to the next stage in the workflow.

In one embodiment, the icons may be fixed in location on the display relative to each other and which are tranversable by a user to via gestural movements of the dental instrument to select a desired icon. Preferably, the currently selected icon is highlighted, enlarged or otherwise modified relative to the other icons to signify its current selection. In one form, the display GUI may comprise icons in a fish-eye format.

In another embodiment, the icons may be moveable in location on the display relative to a selected icon position, the movement being controlled via gestural movements of the dental instrument. Preferably, the icons are movable such that each is movable into the selected icon position for subsequent activation. Preferably, the currently selected icon is highlighted, enlarged or otherwise modified relative to the other icons to signify its current selection. In one form, the display GUI may comprise icons in a rotatable carousel format.

The GUI may comprise a mixture of fixed and movable icon based GUI displays.

The icons may represent data, such as data from electronic patient records, menus, or workflow actions or buttons.

Preferably, a selected icon in the GUI display may be activated via a corresponding activation gesture event or switch activation.

In one embodiment, the dental instrument is provided with an activation switch that may be operated to trigger an activation event to advance the program according to the currently selected icon, and optionally an escape switch that may be operated to revert the program to the previous stage or return to another designated stage in the workflow.

In another embodiment, the dental instrument is provided with virtual switches in the form of a tap detection module that is configured to sense and trigger an activation event in response to a single tap of the dental instrument, and an escape event in response to a double tap of the dental instrument.

The activation and escape events and any other action events may be triggered by any one or combination of the following: real switch activation, virtual switch activation, or designated gesture events.

In a second aspect, the present invention broadly consists in a device driver for controlling an application program running on a host device in response to an input control device in signal communication with the host device, the driver being configured to:
receive from the input control device movement signals sensed by one or more movement sensor(s) onboard the input control device;
monitor the movement signals to detect the occurrence of a gesture event from a set of stored predetermined gesture events; and
output control signals representing detected gesture events to the application program.

In one form, the host device is a computer, and the device driver is a computer device driver.

The second aspect of the invention may have any one or more of the features described in respect of the first aspect of the invention, and by way of example the features defined in relation to the gesture processing module.

In a third aspect, the present invention broadly consists in a method of facilitating user interaction with an application program running on a host device, comprising:
displaying an interactive control GUI on a display associated with the host device;
sensing hand gestural movements of a user via movement sensors onboard a control device held by the user; and
updating the control GUI displayed based on sensed gestural movements.

Preferably, the method further comprises sensing real or virtual switch activation of real or virtual switches onboard the control device and updating the control GUI based on sensed switch activation.

The third aspect of the invention may have any one or more of the features described in respect of the first aspect of the invention.

In a fourth aspect, the present invention broadly consists in a portable handheld dental instrument comprising a main body within which electronic circuitry is mounted and an outer removable cover that is configured to substantially surround the entire or at least a portion of the main body, the cover having a handle portion for gripping by a user and a tool portion extending from an end of the handle portion.

Preferably, the main body is substantially elongate.

Preferably, the removable outer cover is in the form of an elongate sleeve that fits over a substantial portion of the main body. For example, the entire main body or a substantial portion of the main body may be slidably received and retained within the hollow sleeve. The sleeve may be formed from a substantially rigid material, and may be for example molded from plastic.

In one form, the tool portion may be integrally formed with the handle portion. In an alternative form, the tool portion may be releasably mounted to the handle portion.

In use, the removable cover may be disposable and replaceable for each patient, or alternatively may be removed from the main body of the dental instrument for sterilization between each patient.

The removable outer cover may be configured to have a snap-fit, or friction-fit engagement with the main body or may be removably mounted to the main body via an operable latching or locking mechanism.

The tool portion may be a mirror, probe or any other suitable dental tool. In one embodiment, the tool portion is a snap-in mirror that is releaseably mountable to an end of the cover.

The dental instrument may also have any one or more of the features mentioned in respect of the first-third aspects of the invention.

In a fifth aspect, the present invention broadly consists in a removable dental instrument cover for releasably securing to the main body of a control device within which electronic circuitry is mounted, the cover being configured to substantially surround the entire or at least a portion of the main body and having a handle portion for gripping by a user and a tool portion.

The removable dental instrument cover may have any one or more of the features mentioned in respect of the first-fourth aspects of the invention.

In a sixth aspect, the present invention broadly consists in a portable handheld dental instrument comprising a handle portion and a tool portion extending from the end of the handle portion, and the handle portion comprising: a substantially elongate main body within which movement sensors are mounted for sensing movement of the dental instrument in space and generating representative movement signals; and an outer casing that is configured to substantially surround at least a portion of the main body.

Preferably, the outer casing is in the form of an elongate sleeve that fits over at least a substantial portion of the main body.

Preferably, the dental instrument further comprises one or more touch switches mounted to the surface of the main body and wherein the main body and outer casing are configured such that there is an air gap between the touch switches and the inner surface of the outer casing in the vicinity of the touch switches. In one form, the main body is provided with a recessed surface within which the touch switches are mounted so as to provide the air gap between the touch switches and the outer casing. In another form, the outer casing is provided with a recessed surface or reduced thickness in a region aligned with the touch switches of the main body so as to provide the air gap between the touch switches and the outer casing.

Preferably, the outer casing of the dental instrument is resiliently deformable in a region aligned with the touch switches of the main body.

Preferably, the tool portion is a dental mirror that is releasably mounted to the handle portion of the dental instrument.

The sixth aspect may have any one or more of the features mentioned in respect of the first-fifth aspects of the invention.

In a seventh aspect, the present invention broadly consists in a dental charting system, comprising:
a control device configured to be held by a user or which is mounted to another device held by a user;
a movement sensing system configured to sense movement of the control device in space and generating representative movement signals;
a gesture processing module configured to receive and process the movement signals to detect the occurrence of a gesture event from a set of predetermined gesture events, and which generates representative gesture control signals; and
a dental charting application program running on a host device and which provides a graphic user interface on a display associated with the host device for interacting with the application program, the application program being configured to receive and process the gesture control signals to enable interaction with the graphic user interface by the user via hand gesture movements of the control device.

The movement sensing system may be integrated with the control device, such as onboard movement sensors, or external to the control device, or a movement sensing system comprising onboard and external components the co-operate together to sense the movement of the control device.

The seventh aspect of the invention may have any one or more of the features mentioned in respect of the first-sixth aspects of the invention.

Each aspect of the invention may comprise any one or more of the features mentioned in respect of the other aspects of the invention.

The phrase “movement sensor” as used in this specification and claims, unless the context suggests otherwise, is intended to mean any sensing device, system or component that is configured to sense, directly or indirectly, movement or motion of an object and generate representative movement signals of a component or components of the sensed movement such as, but not limited to, position, velocity, acceleration, direction, or the like, and including, but not limited to, inertial sensors such as single, twin, or multi-axis accelerometers and gyroscopes, either alone or in combination, or any other movement sensing device, whether configured to sense movement based on position sensing, orientation sensing, magnetic field sensing, gravitational force sensing, alone or in combination, or any other movement based sensing technology.

The phrase “dental charting application program” as used in this specification and claims, unless the context suggests otherwise, is intended to mean any application program or software configured to run on a computer or other programmable device that is or comprises a graphical dental charting interface, module, function, or workflow for recording observations, treatment plans, or other information about a patient's teeth, whether in the context of dentistry, orthodontics, or other clinical applications. The dental charting application program may be stand-alone or be part of a larger electronic patient records system for use in clinical applications for viewing, updating or otherwise maintaining electronic patient records. By way of example only, some typical electronic patient records systems may provide any of the following features or workflows: patient management, appointment scheduling, electronic clinical records including periodontal and intra and extra oral pathology screens, patient education and 2D or 3D graphical charts, digital radiography including x-ray requests and tracking, risk assessment and related reporting, laboratory and work tracking, paper case notes tracking, DMF recording and reporting, relative value units for evaluating service and provider productivity, waiting lists, and referrals.

The terms “module” and “driver” as used in this specification and claims, unless the context suggests otherwise, is intended to mean a process or function that may be implemented in hardware, software, or any other electronic implementation, and may be standalone or integrated or loaded onto or part of a host device, such as a computer system or other device having computer or microprocessor processing capabilities.

The term “comprising” as used in this specification and claims means “consisting at least in part of”. When interpreting each statement in this specification and indicative independent claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

The invention consists in the foregoing and also envisages constructions of which the following gives examples only.

### BRIEF DESCRIPTION OF THE DRAWINGS

Preferred embodiments of the invention will be described by way of example only and with reference to the drawings, in which:

FIG. 1A shows a schematic diagram of a dental charting system in accordance with a first embodiment of the invention;

FIG. 1B shows a schematic diagram of the signal communication configuration between a portable dental instrument, intermediate interface device, and host device of the dental charting system when the dental instrument is being held by a dentist in use;

FIG. 1C shows the signal communication configuration of FIG. 1B when the dental instrument is not in use and mounted or connected to the intermediate device interface;

FIG. 1D is a schematic diagram showing the electronic components of an intermediate interface device of the dental charting system in accordance with an embodiment of the invention;

FIG. 2 shows a schematic diagram of a dental charting system in accordance with a second embodiment of the invention;

FIG. 3 shows a dental charting system in accordance with a third embodiment of the invention;

FIG. 4 shows a perspective view of a disassembled dental mirror instrument control device in accordance with a first embodiment of the invention showing the main inner body and separate removable outer cover;

FIGS. 5a and 5b show respective side and perspective rendered views of the first embodiment dental mirror instrument of FIG. 4 in an assembled form with the removable cover installed on the main body;

FIG. 6 shows a plan view of the first embodiment dental mirror instrument of FIG. 4;

FIG. 7 shows a cross-sectional view of the first embodiment dental mirror instrument through line AA of FIG. 6;

FIG. 8 shows a cross-sectional view of the first embodiment dental mirror instrument through line BB of FIG. 6;

FIG. 9 shows a front perspective view of an assembled dental mirror instrument control device in accordance with a second embodiment of the invention;

FIG. 10 shows a rear perspective view of the second embodiment dental mirror instrument of FIG. 9 with the main outer casing part of the outer casing assembly omitted from view;

FIG. 11 shows a side elevation view of the second embodiment dental mirror instrument of FIG. 10 with the main inner casing part of the inner casing assembly omitted from view;

FIG. 12 shows a side elevation view of the second embodiment dental mirror instrument of FIG. 11 with the end cap part of the outer casing assembly removed;

FIG. 13 shows a cross-sectional view along the central longitudinal axis of the second embodiment dental mirror instrument of FIG. 9;

FIG. 14 shows a schematic diagram of the main electronic components of a dental instrument control device in accordance with an embodiment of the invention;

FIG. 15 shows a flow diagram of the main signal processing in the dental charting system in accordance with an embodiment of the invention that employs a device driver;

FIG. 16A shows a schematic diagram of the configuration of the sensing axes of inertial sensors of the dental instrument control device in accordance with an embodiment of the invention;

FIG. 16B shows a schematic diagram of a yaw rotation signal representing sensed movement by a gyroscope of the dental instrument control device in space;

FIG. 16C shows a schematic diagram of the rotational pitch signal representing sensed movement by a gyroscope of the dental instrument control device in space;

FIG. 17 is a flow diagram of the processing steps of a gesture processing module of the dental charting system in accordance with an embodiment of the invention;

FIG. 18 shows a flow diagram of the signal processing of a movement processing sub-module of the gesture processing module of FIG. 17 in accordance with an embodiment of the invention;

FIG. 19 shows a flow diagram of the signal processing of a gesture detection sub-module of the gesture processing module of FIG. 17 in accordance with an embodiment of the invention;

FIG. 20 shows a flow diagram of an example state machine for detecting gesture events in the gesture detection sub-module module of FIG. 19;

FIG. 21 shows a screenshot of a dental charting application program where the control GUI is displaying traversable icons in a fish-eye format representing a dental chart;

FIG. 22 shows a screenshot of the dental chart of FIG. 21 including a history sub-chart;

FIG. 23 shows a screenshot of the dental charting application program where the control GUI is displaying movable icons in a rotatable carousel format representing a range of dental service categories;

FIG. 24A shows a flow diagram of the workflow of the application program based on GUI interaction in accordance with an embodiment of the invention;

FIG. 24B shows the workflow diagram of FIG. 24A with the control GUI data and menu icons displayed; and

FIG. 25 shows an alternative form of workflow for the application program in accordance with another embodiment of the invention.

### DETAILED DESCRIPTION OF PREFERRED EMBODIMENTS

1. Overview

The present invention relates to a dental charting system for use in a clinical environment, such as a dental examination or treatment room for viewing, updating and maintaining electronic patient records, and in particular patient dental charts. The dental charting system may be implemented as a standalone system or alternatively a conventional dental charting software application programs may be modified, adapted, or updated or otherwise converted into the dental charting system.

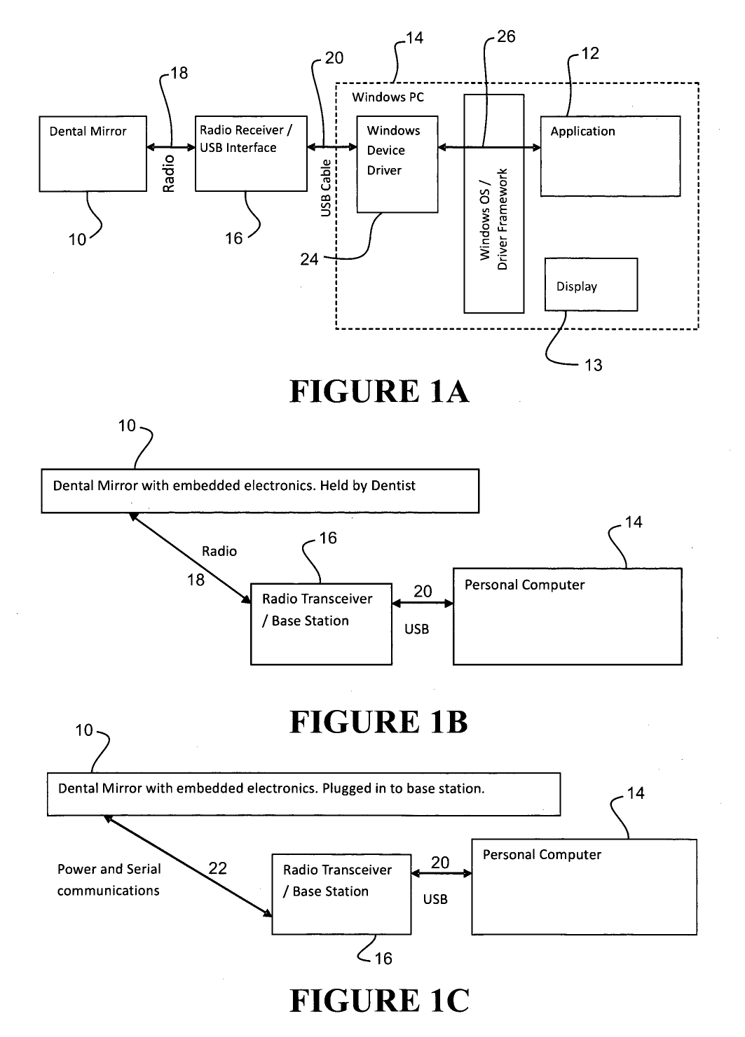

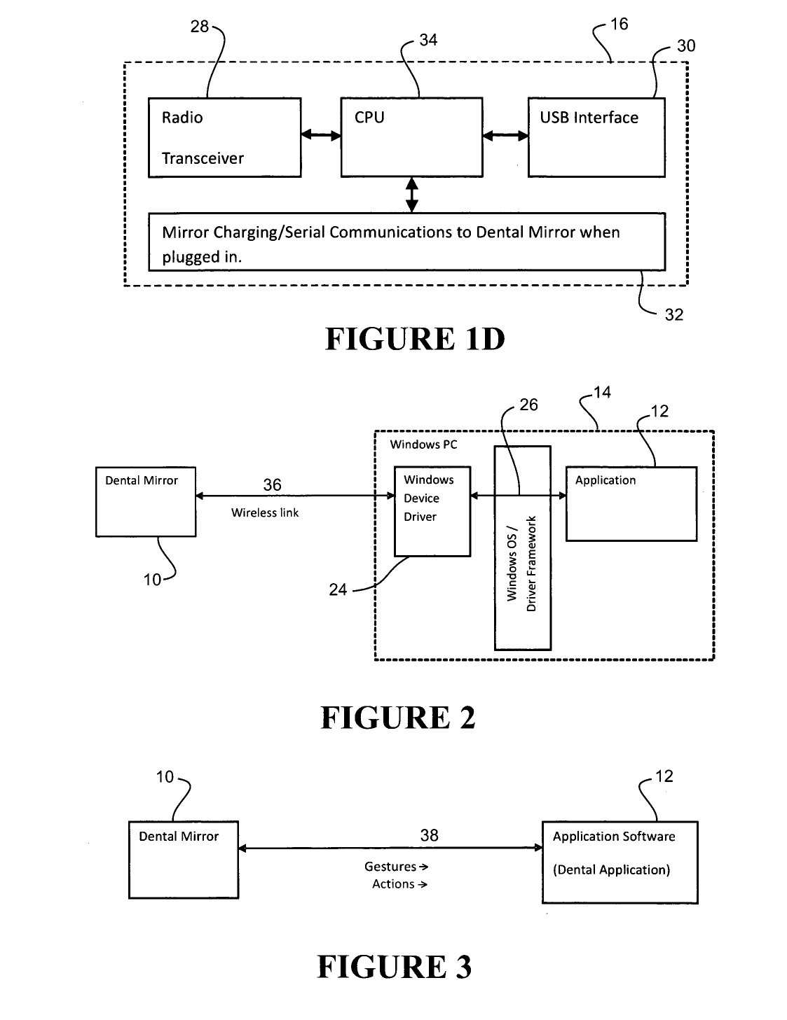

Referring to FIG. 1A, at a general level the dental charting system comprises a control device 10 that may be held by a user, such as a dentist or hygienist in a clinical environment, while examining or treating a patient and which may be moved in 3D space by the user to directly interact with a control graphic user interface (GUI) of a dental charting application program 12 that is running on a host device 14, such as a personal computer having a visual display 13 for displaying the control GUI. In one embodiment, the control device is in the form a portable handheld dental instrument and the following embodiments are described in this context, but it will be appreciated that the features and functionality of the dental instrument may be applied to other forms of the control device. In other embodiments, the control device may be any form of a device that is shaped or configured to be held by a hand of a user including, but not limited to, an elongate control wand. In another embodiment, the control device may be configured to be fixedly or releasably mounted to another handheld device, such as a conventional dental instrument, so that the control device moves with the other handheld device.

In one embodiment, the control GUI is configured to display traversable icons that may be intuitively interacted with based on hand gestural movements of the dental instrument and/or operation of one or more physical and/or virtual switches provided on the dental instrument. For example, in one embodiment the user may use simple directional hand gestures (e.g. left gesture, right gesture, upward gesture, downward gesture) while holding the dental instrument to cause a corresponding traversal of the control GUI icons to enable selection of a desired icon and operate a switch on the dental instrument so as to activate the selected icon to progress to the next stage in the application workflow. More complex gestures (e.g. shaking, dropping, circular movements) and additional switches may be provided to enable further interaction with the control GUI as desired. For example, there may be dedicated switches for activating icons to advance to the next stage in the application program and for escaping to a previous or home menu, or any other desired short-cut switches. Additionally, different types of switch activation (e.g. long press, short press, double press) may cause different program actions.

The dental instrument comprises one or more movement sensors for sensing movement of the instrument in space and generating representative movement signals. In this embodiment, the movement sensors are inertial sensors, although other types of movement sensors could be employed in alternative embodiments. The movement signals are received and processed by a gesture processing module 24 to detect the occurrence of a gesture event from a set of stored predetermined gesture events and which generates representative gesture control signals. The dental charting application program 12 is configured to receive and process the gesture control signals to enable user interaction with the control GUI of the application program 12. Additionally, an action processing module may be provided for receiving and processing switch signals generated by the operation of one or more physical or virtual switches provided in or on the dental instrument and which is arranged to convert the switch signals into action control signals for further interacting with the control GUI of the application program 12.

In a first embodiment in FIG. 1A, the dental instrument 10 is in wireless communication with the host device 14 via an intermediate interface device 16. In this embodiment, the dental instrument 10 is in wireless communication 18 with the intermediate interface device 16. The wireless communication link may use any wireless medium or wireless protocol, including but not limited to customised radio transceiver RF communication, WiFi, Bluetooth, infrared, wireless USB, ANT wireless standard, or any other wireless communication protocol. In one form, the intermediate interface device 16 may be in the form of a base station having a radio transceiver for communicating with a corresponding radio transceiver provided in the dental instrument 10. Additionally, the intermediate interface device 16 may provide a charging platform onto which the dental instrument 10 may be mounted or otherwise connected for hardwired signal communication and/or re-charging when not in use. In this embodiment, the intermediate device interface is in wired communication with the host device 14, although this is not essential and may also be a wireless communication link. As shown, the wired connection may be over a Universal Serial Bus (USB) cable 20 or could be any other connection, such as a serial RS232 connection for example.

The host device 14 may be in the form of a computer system, such as a personal computer, whether a desktop, laptop, or any other programmable device or computing system or device, whether portable or otherwise, that has a processor, memory, user interface, external device interface and an associated visual display 13 for displaying the control GUI of the application program. Conventional computer input devices such as a mouse and/or keyboard may also be provided as supplementary control devices to enable the user to interact with the application program 12.

By way of example, FIG. 1B shows the signal communication between the dental instrument 10, intermediate interface device 16, and host device 14 when the dental instrument is being held and in use by a user. As shown, the movement signals and switch signals from the dental instrument 10 are transmitted over the wireless communication medium 18 to the intermediate interface device 16, and these signals are then transmitted or sent to the host device 14 over a wired communication medium, such as a USB cable or link 20. FIG. 1C shows the signal communication between the devices 10, 16, 14 when the dental instrument is mounted or inserted into the cradle or platform provided by the intermediate interface device 16. In this embodiment, the intermediate interface device 16 and dental instrument 10 are provided with corresponding or complementary electrical connection terminals or contacts for creating a hardwired connection for signal communication 22 between the two devices and recharging of the onboard rechargeable battery supply or other power storage module or system, such as a super capacitor, of the dental instrument 10. It will be appreciated that the dental instrument 10 need not necessarily have rechargeable power supply circuitry and may be provided with a replacable battery supply in the form of an openable or accessible battery compartment having one or more replaceable batteries.

In this first embodiment, the host device is in the form of a personal computer (PC) running a Windows operating system although it will be appreciated that other systems such as the Apple operating system or Linux or any other suitable operating system could alternatively be used. In the Windows PC host device 14, the gesture processing module and any action processing module may be provided in the form of a Windows device driver 24 that is configured to receive and process the movement and switch signals from the dental instrument 10, via the intermediate interface device 16, and translate or convert those signals into respective gesture control signals and action control signals which are sent across the Windows operating system and driver framework as shown at 26 for being received and processed by the control GUI module of the application program 12 to enable the user to interact with the control GUI via gestural movements of the portable dental instrument and switch activation.

In other embodiments, the dental charting system need not necessarily employ a computer as the host device. In such embodiments, the dental charting application program may run on a host device that is in the form of a customised hardware device having a display, processor, memory, and input/output interface for communicating with the control device (e.g. the dental instrument) and for communicating with other external devices such as computers, servers or databases. In such embodiments, the dental charting system may comprise a handheld control device that is in signal communication with the customised hardware device. The customised hardware device may be portable or non-portable unit that may be mounted or placed in a suitable position in the examination room with its display in view of the dentist or user such that they can interact with the GUI of the dental charting application program via gestural movements of the control device. The hardware device is configured to run the application program, display the GUI, and store updated patient data, such as chart information from observations or intended treatment plans, as it is recorded in response to the dentist's interaction with the GUI via the control device. At the end of a session with a patient or after consecutive sessions with multiple patients, the patient data may be uploaded by the dentist to a central database, server or electronic patient record system (EPRS). For example, the hardware device may be connectable to a computer or computer system associated with the central database, server or EPRS via wired or wireless communication to transfer the data. Additionally, the hardware device may be configured to download patient data from the central system prior to a patient session such that the dental charting application program displays the most update dental chart on the GUI when the dentist is examining or treating the patient. It will be appreciated that various synchronisation methods may be employed to update the patient data stored in the central server and the dental charting system. In a clinical environment having multiple examination rooms, each room may have a dental charting system that may be operable to send and receive data to a central server or EPRS, whether onsite or located external to the clinic.

Intermediate Device Interface

Referring to FIG. 1D, the intermediate interface device 16 may comprise a wireless communication module 28 in the form of a radio transceiver for communicating with the dental instrument 10, a USB interface 30 for hardwired communication with the host device 14 and hardwired communication or signal connection module 32 for connecting to the dental instrument when mounted in the device interface. A processor or controller 34 is also provided for interacting and controlling the various modules 28, 30, 32 and this may be in the form of a microprocessor, microcontroller or any other programmable device. It will be appreciated that any one or more of the modules of the intermediate interface device 16 may be integrated. For example, the controller or processor 34 may have an onboard or integrated wireless communication module (radio transceiver) in some embodiments.

The processor 34 of the intermediate interface device 16 may be programmed to carry out various functions and processing. For example, the processor 34 may be configured to handle the protocol conversion from the radio 28 to USB 16 (essentially passing messages unprocessed and vice versa). The processor 34 may also be configured to handle “pairing” of a dental instrument to a given intermediate interface device 16 or host device. For example, a dental instrument may have a unique ID number that is communicated to the intermediate interface device or host device to establish a communication channel so as to prevent interference between signals when multiple dental charting systems are operating in the same vicinity in a clinical environment having multiple examination rooms. The processor 34 may also be configured to control the charging circuitry for the dental instrument and negotiating power requirements with the computer via the USB interface 30. The processor microcode 34 may handle radio interface, including resending missed packets and power management. The CPU microcode also handles USB interface and protocol stack.

PC Device Driver

As discussed, the device driver 24 is programmed to provide an interface between the signals received from the dental instrument and the Windows device driver management (or equivalent for other operating systems). The device driver provides a software interface to a new Windows device in the form of the dental instrument 10, with a customised end message based protocol to provide input to the application program 12. As discussed, the device driver 24 translates the raw and/or pre-processed data (from the inertial sensors and any switches) from the dental mirror 10 into gesture control signals and/or optionally also action control signals for the application software or program running on the PC. The gesture control signals represent gestures carried out by the user's hand or movements related to gestures and these gestures directly control the navigation of the icon-based control GUI of the application program. The application program 12 can also be configured to send status or alert signals to the dental instrument 10 via the device driver 24 to indicate host device status, such as when the PC is put into a sleep state or powered off. In this respect, the communication between the dental instrument and the host device and its application program 12 is bidirectional.

Referring to FIG. 2, a second embodiment of the dental charting system is shown in which there is no intermediate interface device. In this second embodiment, the dental instrument 10 communicates directly with the host device over a wireless communication link 36. It will be appreciated that in alternative forms, the dental instrument might be hardwired via a cable to the host device 14, for example using a USB or a serial port (RS232 or similar). Again, the wireless communication link 36 may be in the form of RF communication, whether Wi-Fi, Bluetooth, or infrared for example.

In both the first and second embodiments of FIGS. 1A and 2, the gesture processing module and/or action processing module are provided in the host device 14, for example in the form of a device driver 24 that translates or converts the raw movement and switch signals from the dental instrument 10 into the gesture control signals and action control signals for interacting with the application program 12. In a third embodiment shown in FIG. 3, the gesture processing module and action processing module may be provided onboard the dental instrument 10. In this embodiment, the dental instrument 10 is configured to send processed gesture control signals and action control signals to the application software running on the host device over any wired or wireless communication link 38 whether directly or via an intermediate device interface.

In a fourth embodiment, the gesture processing module and/or action processing module may be provided in the intermediate interface device 16 such that the raw data or signals sent by the dental instrument 10 are received and processed and converted directly into gesture control signals and action control signals for the host device 14 and its application program 12.

In yet other alternative embodiments, the functionality and processing of the gesture processing module and action processing module may be spread or distributed between the dental instrument, intermediate interface device (if present) and host device such that a mixture of raw, semi-processed and processed signals are communicated between the devices as the case may be.

2. Dental Instrument

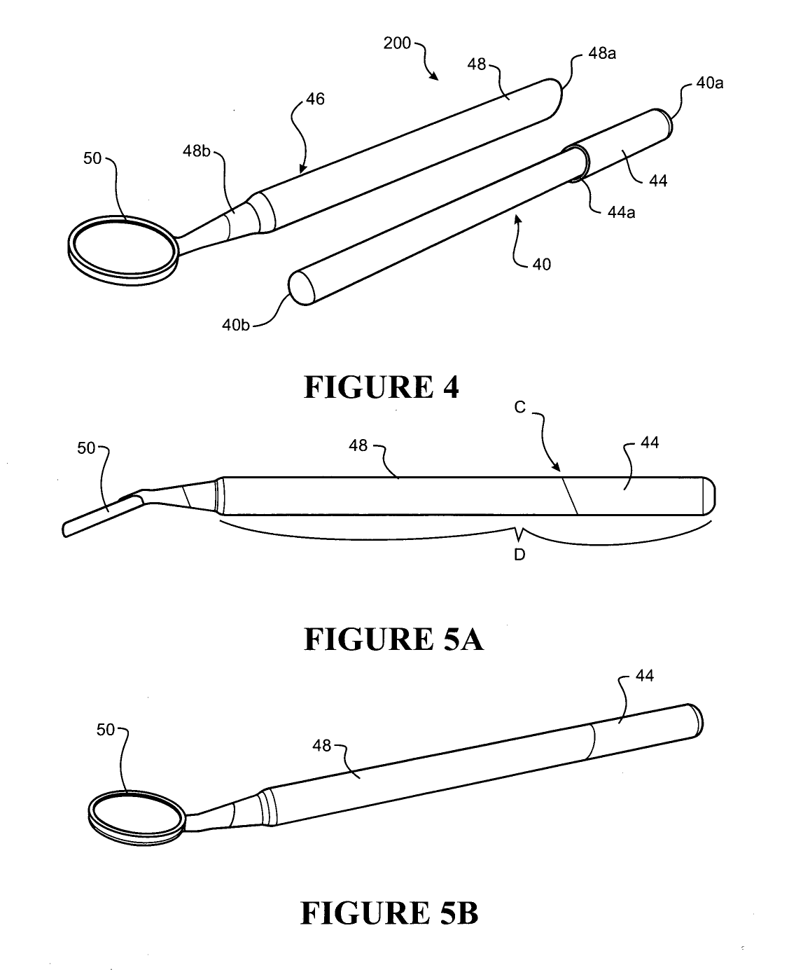

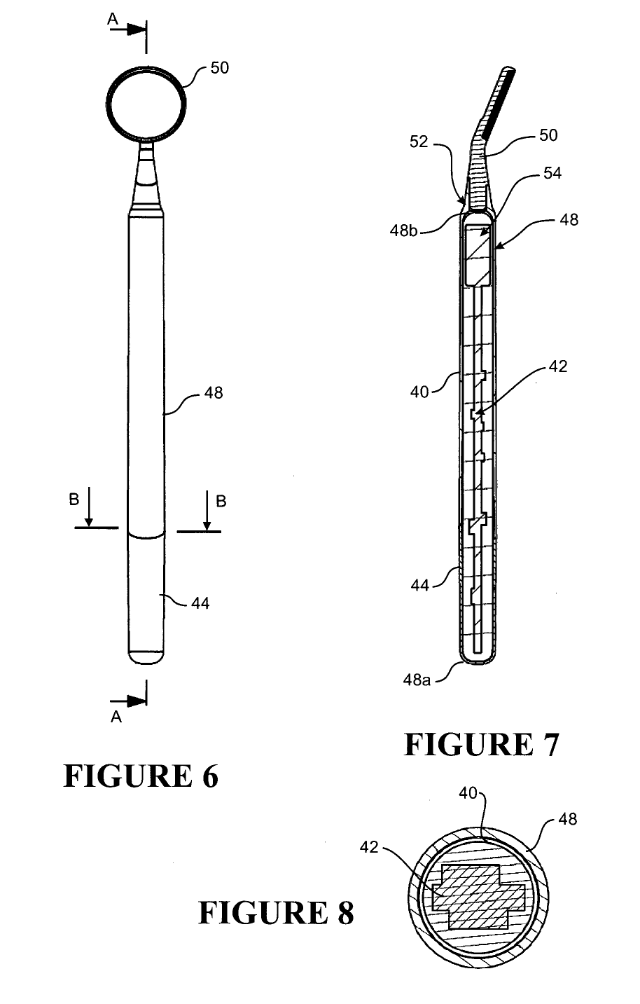

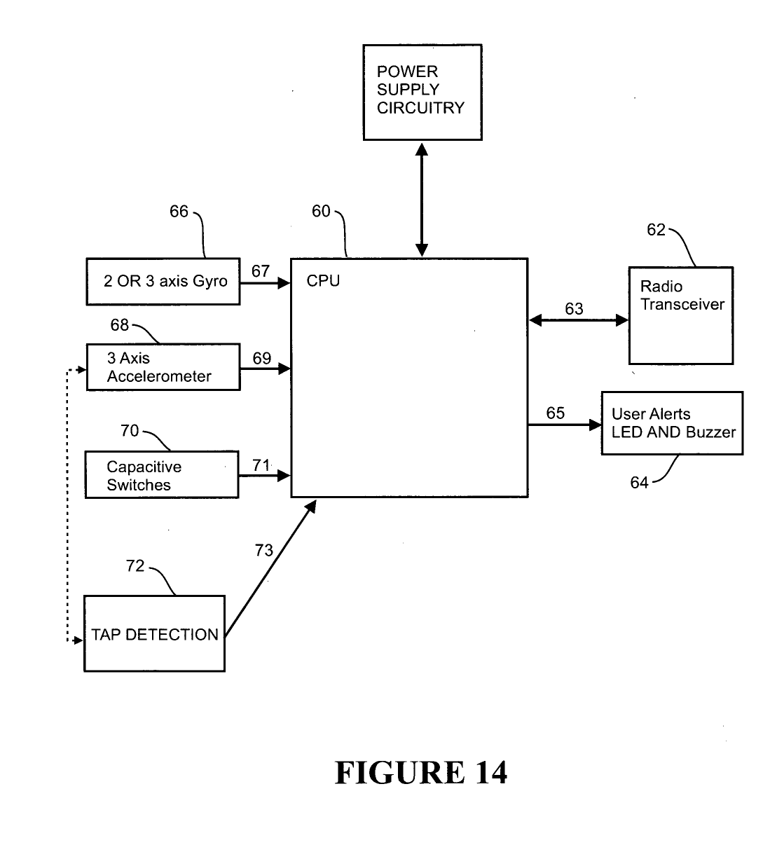

Example embodiments of the dental instrument control device 10 will be described with reference to FIGS. 4-13. Firstly, the physical housing and construction of a first embodiment dental instrument control device 200 and second embodiment dental instrument control device 300 will be described with reference to FIGS. 4-13. Secondly, the primary electronic circuitry, components and modules of the dental instrument control device 10 will be described with reference to FIG. 14.

The main features of the portable handheld dental instrument are that it is portable and provides a handle portion for the dentist to grip along with a tool portion, such as the mirror, probe or any other dental tool, and which houses powered electronic circuitry inside the dental instrument. The type of power supply circuitry and whether or not electrical contacts, terminals or connectors are provided for communicating with the internal circuitry may be varied as desired.

First Embodiment Dental Instrument

With reference to FIG. 4, the first embodiment the dental instrument 200 comprises a two-part construction. The first part of the dental instrument is a main body 40 that extends between a first end 40a and second end 40b and which encloses the electronic circuitry, which is for example provided on a printed circuit board (PCB) 42 (see FIG. 7). In this embodiment, the main body 40 is a substantially hollow body having a circular cross section along its length, although this profile may be otherwise shaped along its length if desired. In this embodiment, a lower portion of the main body toward the first end 40a is provided with an outer sheath 44, which may be moulded over the main body 40 or otherwise integrally formed or fixed to the main body. The second part of the dental instrument is a removable outer cover 46 that is arranged to be securely attached to the main body 40 such that it substantially surrounds the entire or at least a substantial portion of the main body 40.

In this embodiment, the removable outer cover 46 comprises an elongate handle portion 48 that extends between a first end 48a and a second end 48b, and a tool portion 50 that extends from the second end 48b of the handle portion 48. The handle portion 48 is substantially hollow, and is shaped and sized as an outer sheath for covering the main body 40. For example, the second end 40b of the main body 40 may be inserted into the first open end 48a of the handle portion 48 of the removable outer cover such that it may be slidably received and retained within the handle portion 48 as shown in FIGS. 5A and 5B. When the removable cover 46 and main body 40 are assembled together as shown in FIGS. 5A and 5B, the first end 48a of the handle portion 48 of the removable cover abuts the edge or rim 44a provided by the lower outer sheath 44 of the main body 40 as shown by arrow C in FIG. 5A. As shown, a substantial portion of the handle portion 48 has a cross-sectional shape that is arranged to compliment the shape of the main body 40. Further, the diameter of the handle portion 48 in the region that receives the main body 40 is substantially equal to the diameter of the integrally formed or attached outer sheath portion 44 of the main body such that the joint between the two components is flush providing a smooth and uniform shaped outer gripping surface for a user to grip as shown at D in FIG. 5A.

The tool portion 50 of the removable cover 46 may be integrally formed or fixed with the handle portion 48 or alternatively removably mounted or detachable such that different tool portions or components may be mounted to the handle portion as desired. In this embodiment, the dental instrument is in the form of a dental mirror and the tool portion is a snap-in mirror that is configured for releasable engagement with a retaining formation 52 provided at the second end 48b of the handle portion 48.

The main body and removable cover are preferably modelled from plastic but may also alternatively be formed from any other suitable rigid material, such as aluminium or carbon fibre. The outer sheath portion 44 may be integrally moulded plastic or a rubber sheath for example.

In use, the removable cover 48 may be removed from the main body 40 and then cleaned and sterilized between patients and then reassembled with the main body 40. Alternatively, the removable cover 48 may be disposable and for one time use only. In this embodiment, the switches employed on the dental instrument are preferably tap detection or touch switches, or any other type of switch that may be mounted to or within the main body 40 and which may co-operate with the removable cover. The removability of the cover allows the main body 40 with its electronic circuitry to be separated from the cover part of the instrument that is in contact with the dentist's hands and patient's mouth. The cover may therefore be separately sterilised or disposed of on its own, without the need to submit the electronics in the main body through this process.

The main body 40 toward the first end 40a may also be provided with electrical terminals, contacts, or a socket for connecting to corresponding terminals, contacts, or a plug of the intermediate interface device when mounted in the interface or cradle or otherwise connecting via cable to a host device for data transmission, programming, and/or battery recharging or the like. As shown in FIG. 7, the electronic circuitry of the PCB 42 is supplied power by a rechargeable battery package 54 mounted at or toward the second end 40b of the main body 40.

Second Embodiment Dental Instrument

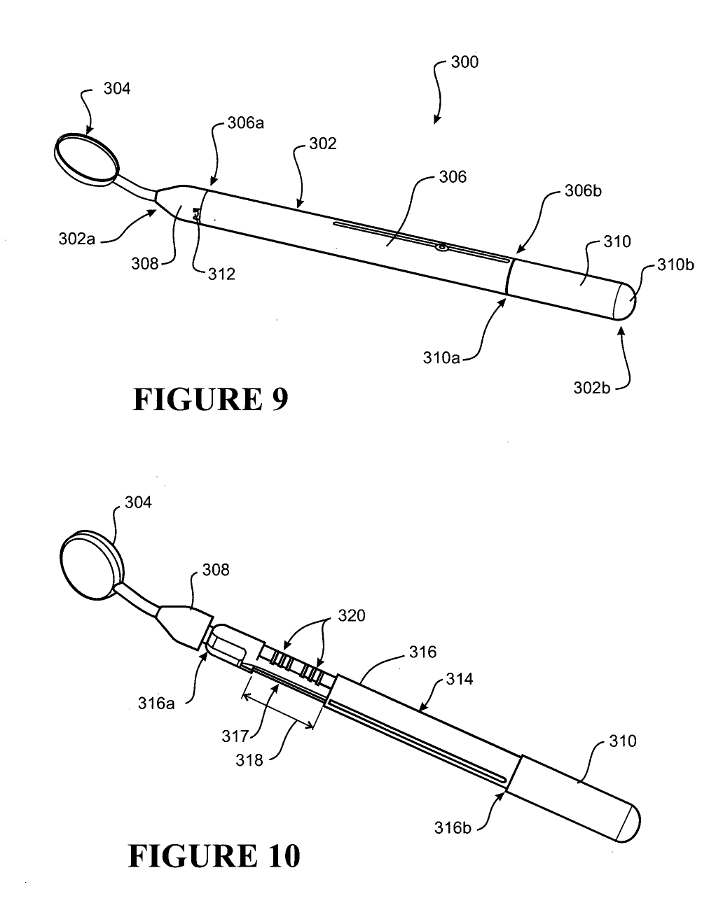

With reference to FIG. 9, the second embodiment dental instrument 300 comprises an elongate outer casing assembly 302 extending between a first end 302a and a second end 302b. A tool part or portion 304, such as a dental mirror or other tool, is fixed or releasably mounted at or toward the first end 302a of the outer casing assembly 302. In this embodiment, the outer casing assembly is substantially cylindrical in shape along a substantial portion of its length and is configured for gripping by a hand of a user. The outer casing assembly comprises a central or main outer casing part 306 in the form of a hollow cylinder or sheath or outer cover extending between a first end 306a and second end 306b. The main outer casing part provides the handle or gripping portion of the dental instrument 300. In this embodiment, the main outer casing part 306 is formed from plastic via blow-molding but could alternatively be injection molded or formed from another suitable material if desired. By way of example, any suitable blow-moldable plastics may be used including, but not limited to, polypropylene, polyethylene, high-density polyethylene (HDPE), polyvinyl chloride (PVC), and polyethelyne terephthalate (PET). The main outer casing part 306 is of a thickness and/or type of plastics material that provides sufficient rigidity to substantially maintain its shape when being held. The outer casing assembly further comprises a tool mounting part or connector part 308 extending from or connected to the first end 306a of the main outer casing part 306 and an end cap part 310 extending from or connected to the second end 306b of the main outer casing part 306.

In this embodiment, the connector part 308 comprises a cylindrical base portion that terminates with a frusto-conical portion, and is configured to releasably retain a tool part 304 via a screw-thread configuration. For example, the connector part 308 comprises a centrally extending aperture having a screw-threaded internal surface that is configured to receive and retain a complementary threaded engagement portion 304a (see FIG. 13) of the tool part 304. The connector part 308 is coupled or connected to the first end 306a of the main outer casing part 306 by one or more clips 312, or alternatively a snap-fit, screw-thread, friction fit, or any other suitable connection configuration.

In this embodiment, the end cap part 310 has an open first end 310a and an enclosed second end 310b. The enclosed second end 310b is hemi-spherical in shape in this embodiment but may be flat or any other shape if desired. The end cap part is substantially hollow and is configured to provide a battery compartment within which a removable and replaceable battery of the dental instrument 300 is be received and retained.

Referring to FIG. 10, the dental instrument 300 further comprises an elongate inner casing assembly or main body generally indicated at 314 that is mounted inside or within the outer casing assembly 306. The inner casing assembly 314 houses the electronic circuitry of the dental instrument 310. The inner casing assembly comprises a main inner casing part 316 that extends between a first end 316a and second end 316b. The main inner casing part 316 is substantially cylindrical in shape and in this embodiment is formed from plastic or another suitable material by over-molding about the inner components of the dental instrument 300. The plastics material may be any suitable plastics material including the examples mentioned above in relation to the outer casing part. The predominant cross-sectional outer surface diameter along the length of the main inner casing part 316 is slightly smaller than the corresponding inner surface diameter of the main outer casing part 306 such that outer casing part fits snugly over or around the inner casing part 316 in a sheath-like and abutting fashion.

In this embodiment, the main inner casing part 316 is provided with a recessed surface 317 or portion of reduced diameter relative to the remaining predominant or main portions cylindrical outer surface as shown between the arrows at 318. This recessed surface 317 does not abut the inner surface of the main outer casing part 306. The recessed surface is provided toward the first end 316a of the main inner casing part 316, which corresponds to the tool part 304 end of the dental instrument 300. This embodiment of the dental instrument 300 comprises one or more capacitive touch-switches 320 for operation by the user's fingers in use. Typically, the touch-switches comprise an arrangement of annular or ring-shaped electrodes. In this embodiment, the touch-switches are mounted to or about or provided in the recessed surface 317 of the inner casing part 316 such that there is an air gap between the surface of the touch switches (e.g. the electrodes) and the inner surface of the main outer casing part 306 in their vicinity as shown more clearly in FIG. 13. The air gap assists in minimising or reducing the incidents of accidental or unintentional actuation or activation of the touch switches when the dental instrument is in use.

With the recessed touch switches, the user must squeeze or apply sufficient pressure to flex the outer casing part 306 inwardly in the vicinity of the touch switches to actuate or trigger them, which requires a conscious effort. To accommodate this, the main outer casing part 306 is sufficiently thin or resiliently deformable at least in the region of the touch switches to enable flexing or bending of the casing under the pressure or force of a user's fingers and/or thumb. The main outer casing part 306 may have a uniform thickness along its length or alternatively a non-uniform thickness comprising a reduced thickness in the region associated with the touch switches. In this embodiment, the thickness of the plastic or wall of the main outer casing part 306 in the vicinity of the touch switches is preferably in the range of about 0.1 mm to about 1 mm, more preferably about 0.2 mm to about 0.8 mm, or even more preferably about 0.4 mm to about 0.6 mm. In this embodiment, the air gap between the touch switches and the inner surface of the main outer casing part 306 is preferably in the range of about 0.1 mm to about 2 mm, more preferably about 0.5 mm to about 1.5 mm, even more preferably about 1 mm. It will be appreciated that robust touch switch configuration which allows for easy user activation but minimises accidental or inadvertent activation depends on a combination of factors, including the type of wall or plastic material, thickness of the wall, and the air gap distance between the touch switches and the wall. Typically, a larger air gap distance requires the wall of the main outer casing part in the region of the switches to have a higher degree of flex or resilient deformability (e.g. via thinner of softer plastic for example) so that the user can deform with their finger or thumb the wall in the vicinity of the touch switches into the air gap toward the touch switches with a level of displacement that is sufficient to trigger or activate the touch switches. A smaller air gap distance requires the wall of the main outer casing part in the region of the switches to have a lower degree of flex or resilient deformability (e.g. via thicker or stiffer/harder plastic for example) as the wall in the vicinity of the touch switches need only deform a smaller amount under the pressure or force of the user's finger or thumb for switch activation.

In an alternative arrangement, it will be appreciated that the air gap configuration in the vicinity of the touch switches may be provided in other ways. For example, in an opposite configuration the main inner casing part may alternatively have a substantially uniform diameter along its length without a recessed portion and with the touch switches being mounted to or provided on that surface. The main outer casing part may be provided with a recessed inner surface or portion or region of reduced thickness in the vicinity of the touch switches such that an air gap is provided between the switches and the inner surface of the main outer casing part.

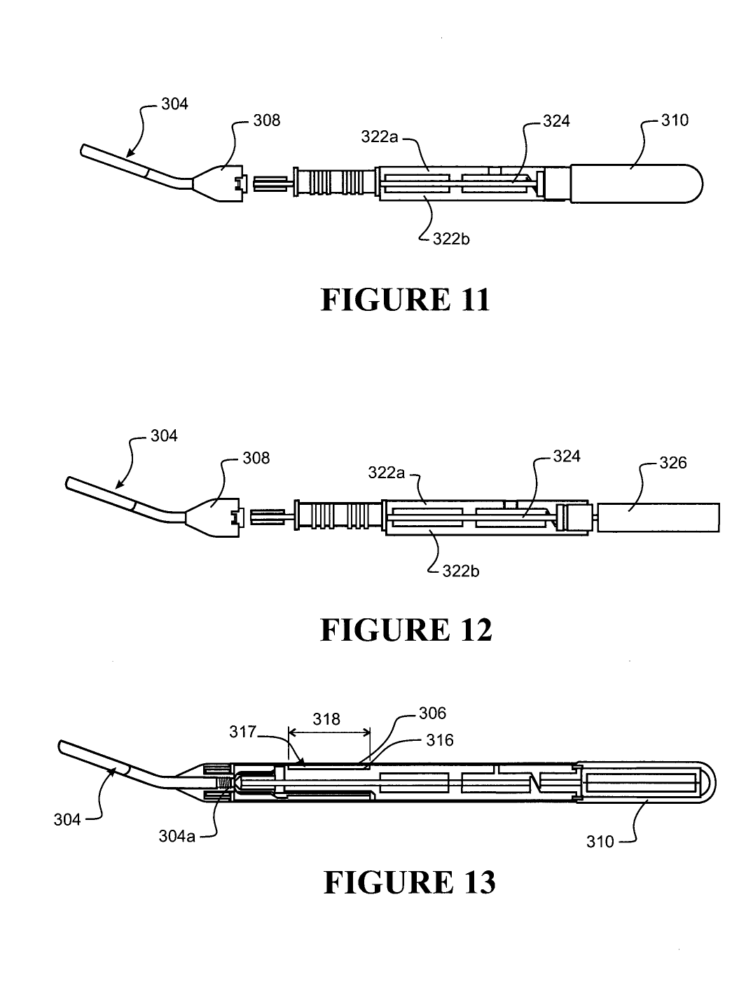

Referring to FIG. 11, the inner casing assembly further comprises upper 322a and lower 322b spine or frame parts between which a PCB 324 with electronic circuitry of the dental instrument 300 is mounted. The upper 322a and lower 322b frame parts are mounted or enclosed within the over-molded main inner casing part 316.

Referring to FIG. 12, a battery or battery package 326 is shown connected to the end of the PCB 324 and which is received and retained within the battery compartment formed within the end cap part 310 of the outer casing assembly. The battery pack 326 may be rechargeable or replaceable.

The outer casing assembly is preferably formed from a sterilisable plastic or other material. Additionally or alternatively, one or more of the outer casing assembly parts may be configured to be disposable or for one-time only use.

Other Alternative Constructions of the Dental Instrument

It will be appreciated that any other dental instrument construction may alternatively be employed. The construction need not be a two-part construction with a removable cover like dental instrument 200 or comprise inner and outer casing assemblies like dental instrument 300. In alternative embodiments, the dental instrument may be a single integral body which surrounds the internal electronic circuitry and having a handle portion for gripping and a tool portion or part. In other alternative embodiments, the dental instrument may comprise a single handle portion that encloses the electronic circuitry and which provides a mounting for releaseably mounting a selection of detachable tools, such as mirrors, probes or any other dental tools.

Electronic Components and Circuitry

Referring to FIG. 14, the electronic circuitry provided on the PCB of the dental instrument 10 comprises a main controller 60 that may be in the form of a CPU, microprocessor, microcontroller, or any other programmable device and which is arranged to operate and coordinate the processing and functionality of the other modules and electronic circuitry components of the instrument 10. The controller 60 may have an integrated or separate wireless communication module 62 for communicating data and signals directly to the host device 14 or indirectly via the intermediate interface device 16. As previously discussed, the wireless communication module may be any form of wireless communication protocol, and may be in the form of a radio transceiver, or may implement Wi-Fi or Bluetooth communication or in an alternative form infrared communication may be provided. Communication signals being sent and received via the wireless communication module 62 are shown at 63. A user alerts module 64 is provided for alerting the user of the dental instrument of the status of the system. The user alerts may comprise visual, audible or tactile output devices. In one form, the user alerts may comprise lights such as LEDs and/or an audible output such as a buzzer, and/or vibration module for alerting the user to the status of the system or other alerts and system feedback. The user alert module 64 is controlled by user alert control signals 65 from the controller 60.

As previously discussed, the dental instrument comprises inertial sensors for sensing gestural movements of the instrument in space. In this embodiment, the inertial sensors comprise a gyroscope sensor 66 and an accelerometer sensor 68. The gyroscope sensor 66 is mounted within the dental instrument on the PCB 42 and is configured to sense rotation of a reference axis of the dental instrument with reference to one or more instrument reference planes and generate representative instrument rotation signals 67 as will be described in further detail later. The accelerometer sensor 68 is mounted to or within the dental instrument and is configured to sense the orientation of the reference axis of the dental instrument with respect to gravity and generate representative orientation signals 69 as will be explained in further detail later. The gyroscope signals 67 and accelerometer signals 69 are received by the main controller 60 and either sent in raw form over the wireless communication module 62 to the host device or alternatively may be pre-processed and then sent to the host device.

The electronic circuitry may further comprise a switch module 70 that comprises one or more physical switches that are operable by the fingers of a user holding the dental instrument to generate switch signals representing the operation or activation of the one or more switches. The switches may be in any suitable form and may be mounted on the housing or casing of the dental instrument in any suitable location for operation by the fingers and/or thumb of a user gripping the dental instrument. By way of example only, the physical switches may be touch switches (such as capacitive switches) or mechanical switches (such as micro-switches), or any other suitable switching components. In one embodiment, the switches are mounted to the main body or inner casing part and covered by the removable cover. Each switch is operable via a finger or thumb press in the region of the cover associated with the switch location. The switch signals 71 are received by the main controller 60 and either sent in raw form or alternatively processed and then sent in pre-processed form to the wireless communication module 62 for transmission to the host device either directly or indirectly via the intermediate interface device 16.

In other embodiments, the switch module 70 may be in the form of virtual switches that are triggerable by movement of the instrument. For example, the virtual switch may be in the form of a tap detection module 72 that is configured to detect “single taps” or “double taps” and generate representative switch signals 73. In one form, the tap detection module may be integrated with the accelerometer sensor such that tap detection is based on the accelerometer signals or alternatively the tap detection module may be performed in the main controller 60 based on the raw accelerometer signals 69.

As will further be explained in detail later, the accelerometer is preferably a three-axis accelerometer that measures acceleration in three orthogonal axes. The gyroscope sensor 66 in this embodiment is a two-axis gyroscope that is positioned to sense rotation of a reference axis of the dental instrument with reference to two perpendicular instrument reference planes. However, alternatively it will be appreciated that three-axis gyroscope could alternatively be used to provide a measurement in an additional rotational direction for increasing the types of gestures that may be detected.

3. Gesture Control Signals and Action Control Signals

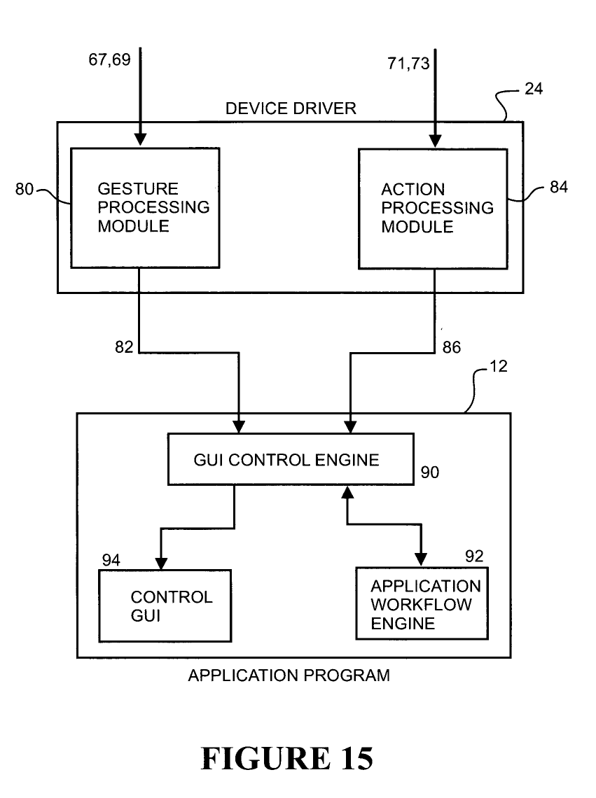

Referring to FIG. 15, the processing of the movement signals 67,69 from the inertial sensors 66,68 and switch signals 71,73 from the physical or virtual switches will be described in further detail. In the embodiment of FIG. 1A, the dental charting system is provided with a device driver 24 that is configured to run on the host device 14. The device driver comprises a gesture processing module 80 that is configured to receive and process the movement signals 67,69 from the inertial sensors to detect the occurrence of a gesture event from a set of predetermined gesture events, and which generates representative gesture control signals 82 for the application program 12. Additionally, the device driver 24 may comprise an action processing module 84 that is configured to receive and process the switch signals 71,73 from the physical and/or virtual switches of the dental instrument to detect the occurrence of action events from a set of predetermined action events, and which generates representative action control signals 86 for the application program 12.

It will be appreciated that the gesture processing module 80 and action processing module 84 need not necessarily both be provided in the device driver 24 of the host device and may alternatively be implemented in the dental instrument or their functionality may be spread between the dental instrument, any intermediate interface device 16, and the host device 14 as previously explained.

The application program 12 comprises a GUI control engine 90 that is configured to receive and process the gesture control signals 82 and action control signals 86. In response to the control signals 82,86 the GUI control engine interacts with the application workflow engine 92 which controls the flow and operation of the application program and updates the corresponding control GUI display according to the status of the application workflow. For example, the application workflow engine 92 retrieves data and electronic patient records for display by the control GUI depending on the status of the workflow, updates the control GUI displays, and updates electronic data based on the user's interaction with the control GUI 94 as will be explained in further detail later.

The gesture processing module and action processing module will now each be explained in further detail.

Gesture Processing Module

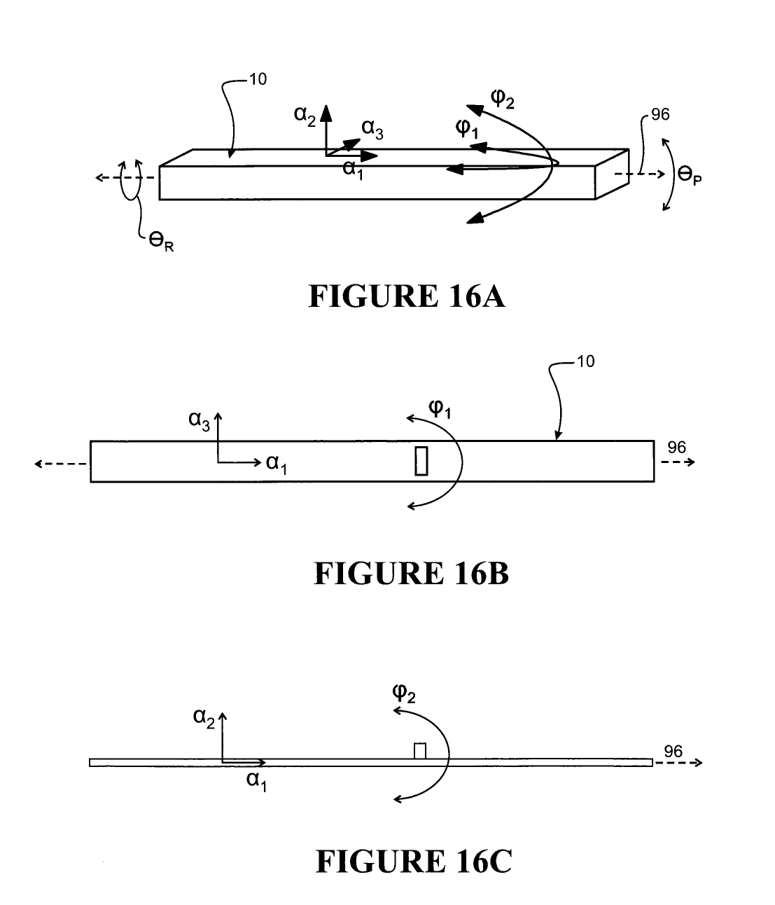

Referring to FIGS. 16A-16C, the configuration of the accelerometer sensor 68 and gyroscope sensor 66 in the dental instrument 10 will be described in regard to the gesture processing module 80. In this embodiment, the dental instrument has a predefined reference axis 96. In this embodiment, the reference axis 96 is aligned or parallel with the longitudinal axis of the elongate dental instrument, although the orientation of the reference axis can be selected as desired. The inertial sensors 66,68 generate movement signals 67,69 that may be processed into signals that represent the rotation of the reference axis 96 with reference to one or more global reference planes oriented with respect to gravity. For example, the gyroscope signals represent the rotation of the instrument relative to itself, and these signals may be processed to represent rotation of the instrument relative to gravity based on the accelerometer signals. In this embodiment, the global reference planes comprise a horizontal plane with reference to gravity and a vertical plane with reference to gravity. Other embodiments of the invention may only detect rotation of the reference axis 96 in one of these horizontal or vertical reference planes or in other reference plane(s) having another orientation with respect to gravity depending on the number and type of gestures that are to be detected to enable the required interaction with the application program.

In this embodiment, the accelerometer sensor 68 may be a three-axis accelerometer that is configured to measure acceleration in three orthogonal axes, α₁, α₂, and α₃, as shown in FIG. 16A. Preferably, at least one of the accelerometer axes α₁, α₂, α₃ is co-aligned or parallel with the reference axis 96 of the dental instrument 10. The accelerometer signals of three-axes of the accelerometer sensor 68 may be processed to calculate the orientation of the reference axis 96 of the instrument with respect to gravity.

The gyroscope sensor 66 may be in the form of a two-axis gyroscope that is configured to sense rotation of the reference axis 96 in two orthogonal directions φ₁, φ₂. The two rotation directions φ₁ and φ₂ are located in respective perpendicular instrument reference frames. Referring to FIG. 16B, the first rotation direction φ₁ measures rotation of the reference axis 96 in a first instrument plane that is parallel or aligned with axes α₁ and α₃ of the accelerometer sensor 68. Referring to FIG. 16C, the second rotation direction φ₂ measures rotation of the reference axis 96 in a second instrument plane that is parallel or aligned with axes α₁ and α₂ of the accelerometer sensor 68. In this embodiment, both the first and second instrument reference planes are co-aligned with the reference axis 96 such that the reference axis 96 lies in the intersection of the two planes. In operation, the gyroscope sensor is configured to sense the rotation φ₁ and φ₂ of the reference axis 96 in the first and second instrument reference planes and generates representative instrument rotation signals. In this embodiment, the instrument rotation signals for φ₁ and φ₂ represent a continuous measurement of change of rotation (angular velocity) but may alternatively be configured or processed to generate an absolute rotation angle relative to a zero reference if desired. It will be appreciated that the gyroscope sensor may alternatively be a three-axis gyroscope that is configured to sense rotation with respect to a third instrument reference plane if desired.

In this embodiment, the first direction of rotation φ₁ is sensing the local yaw movement of the reference axis 96 and the second direction of rotation φ₂ is sensing the pitch of the dental instrument with respect to a local reference frame of the instrument itself. However, it will be appreciated that the dental instrument may be held in various orientations with respect to gravity and therefore in this embodiment the local yaw φ₁ and pitch φ₂ signals are adjusted according to the orientation of the dental instrument with reference to gravity as will be explained next with reference to the gesture processing module. In other words, the instrument rotation signals φ₁,φ₂ generated by the gyroscope sensor are oriented with respect to gravity based on the accelerometer signals from the accelerometer sensor to provide global pitch and yaw signals for subsequent gesture detection.

It will be appreciated that the gyroscope and accelerometer sensors need not necessarily be mounted to be aligned with a reference axis or each other. A calibration module may be provided that is configured to determine the relationship between the sensors (and their sensor axes) with respect to each other and the control device, and adjust or convert the sensor signal outputs accordingly. Typically, this calibration may be performed automatically at device initialisation or during an initial device configuration after construction. The calibration module may be provided in any suitable part of the system, or calibration may be performed on the system prior to its use.

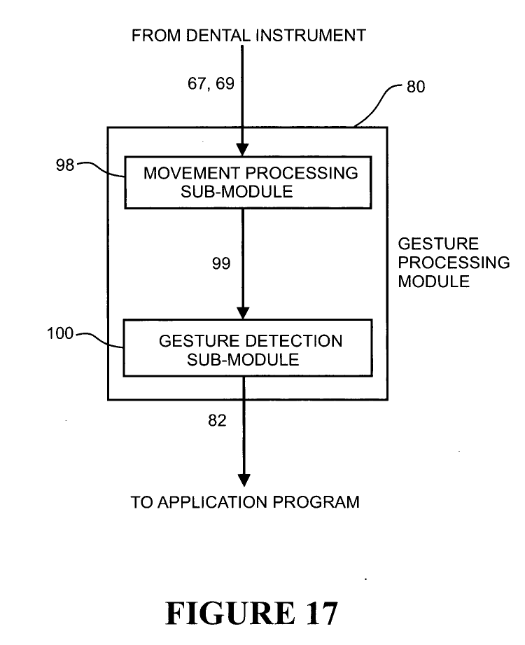

Referring to FIG. 17, in this embodiment the gesture processing module 80 comprises a movement processing sub-module 98 and a gesture detection sub-module 100. The movement processing sub-module 98 is configured to receive and process the raw accelerometer and gyroscope signals 67,69 to generate global rotation signals 99 representing the rotation of the reference axis 96 of the dental instrument 10 with reference to the vertical and horizontal global reference planes oriented with respect to gravity. The gesture detection sub-module 100 receives and processes the global rotation signals 99 from the movement processing sub-module 98 to detect the occurrence of gesture events, and generates representative gesture control signals 82 representing any detected gesture events for sending to the host device and/or application program. Each of these sub-modules 98,100 will now be described in further detail.

Movement Processing Sub-Module

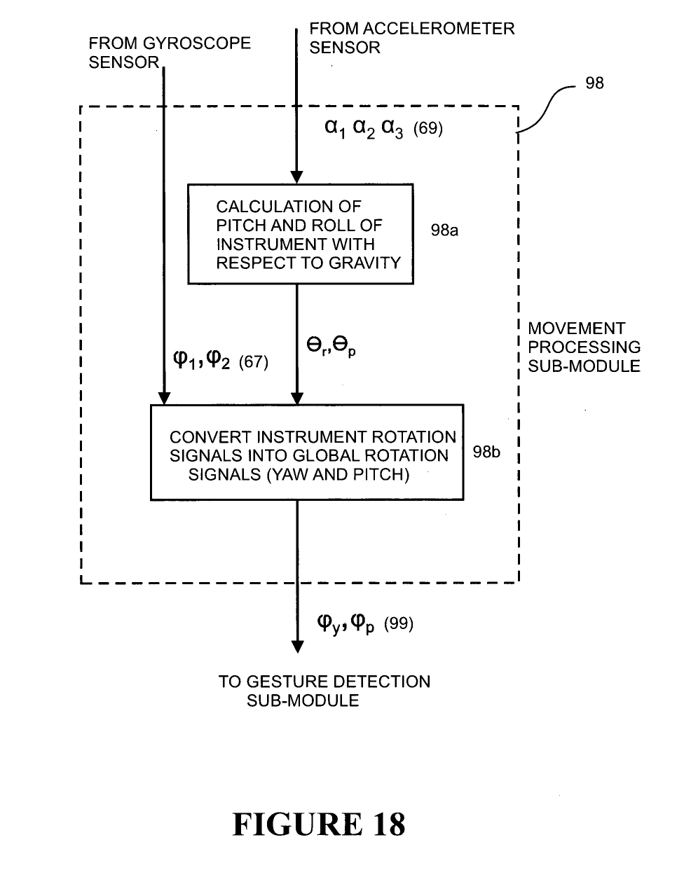

Referring to FIG. 18, the signal processing of the movement processing sub-module 98 will now be described further. First, the sub-module 98 receives and processes the accelerometer signals 69 at step 98a to generate signals representing the roll θᵣ and pitch θₚ. The roll θᵣ represents how the dental instrument or reference axis 96 is twisted with respect to gravity and the pitch θₚ represents how far the dental instrument or reference axis 96 is tilted upward or downward relative to gravity, as shown graphically in FIG. 16A. The following equations are used to calculate the signals:

$${θ}_{r} = {tan}^{- 1} \frac{{α}_{3}}{{α}_{2}}$$

$${θ}_{p} = {tan}^{- 1} \frac{{α}_{1}}{{α}_{3} sin {θ}_{r} + {α}_{2} cos {θ}_{r}}$$

Second, the sub-module 98 converts the instrument rotation signals 67 (i.e. rotation signals of instrument relative to itself) from the gyroscope sensor 66 into global rotation signals φ_{y} and φₚ (i.e. rotation signals of instrument relative to gravity) which represent rotation (in angular velocity) of the reference axis 96 of the dental instrument with respect to the respective horizontal and vertical global reference planes based on the pitch and roll signals θᵣ, θₚ. In this embodiment, the global yaw signal φ_{y} represents the rotation of the reference axis 96 of the dental instrument in the horizontal global reference plane with respect to gravity and the global pitch signal φₚ represents the rotation of the reference axis in the vertical global reference plan with respect to gravity. The global pitch and yaw signals are calculated with the following equations:

φ_{y}=φ₁ cos θᵣ−φ₂ sin θᵣ

The polarity of the global pitch φₚ and global yaw φ_{y} signals 99 is defined or configured to represent the respective direction of rotation in their respective vertical and horizontal global reference planes. For example, a positive global yaw signal may define a rotation in the left direction and a negative global yaw signal may define a rotation in the right direction or vice versa. Likewise, a positive global pitch signal may represent a pitching upward of the reference axis while a negative global pitch signal may represent a pitching or tipping downward of the reference axis of the dental instrument, or vice versa as desired.

It will be appreciated that global yaw signal φ_{y} (in angular velocity) may be integrated to calculate the yaw rotation angle θ_{y} using the following equation:

$${θ}_{y} = \int {cos}^{- 1} ( \frac{cos {ϕ}_{y} - {sin}^{2} {θ}_{p}}{{cos}^{2} {θ}_{p}} )$$

The integration may be adjusted by the pitch, analogously to calculating the change of longitude on the Earth (e.g. θ_{y}) given a rotation angle relative to the Earth's centre. These calculations have provided the reference frame for detecting gestures in the gesture detection sub-module 100 relative to gravity and for determining the angles at which the device is pointing.

Gesture Detection Sub-Module

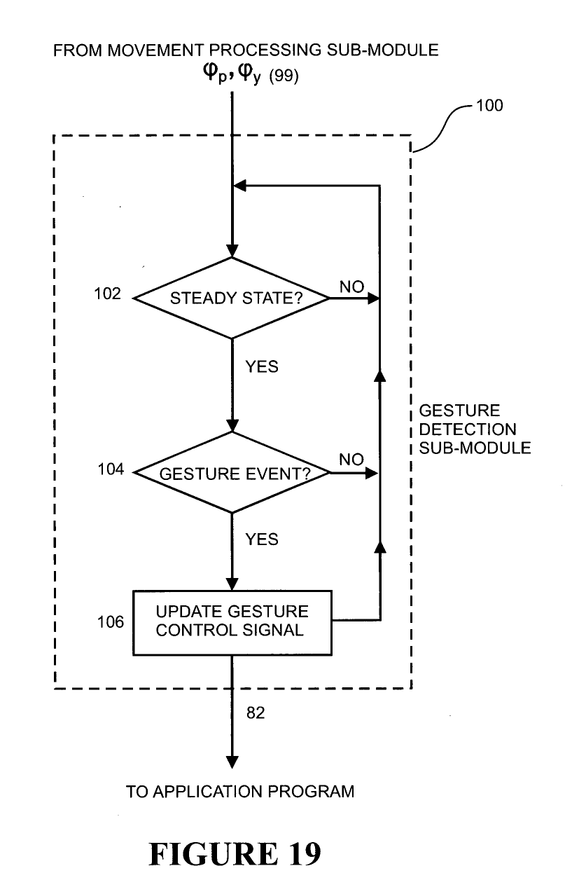

Referring to FIG. 19, the gesture detection sub-module comprises a set of predetermined gesture events for detecting, each gesture event being defined by a predetermined fluctuation profile or changes in the level of one or more of the global rotation signals. In one embodiment, each gesture event is defined by a movement sequence that is defined by the change in magnitude of one or more of the global rotation signals relative to predefined signal thresholds and/or timing profiles.

The gesture detection sub-module 100 is configurable to sense any number of different types of hand gestures that may made while the user is holding the dental instrument. Each gesture event that is to be detected causes a corresponding intuitive interaction or control of the displayed control GUI in the application program. In other words, the control GUI in the application program is configured to react to gesture events in a corresponding intuitive way that matches the nature of the user's hand gesture. By way of example only, the gestures may be simple directional gestures such as left or right movement gestures or up and down movement gestures. Additionally or alternatively the gestures may be more complex such as angular movement gestures, shaking, circular movements, twisting or pivoting gestures, tipping, or any other type of gesture that may be performed with the dental instrument. Each of these different types of hand gestures may be detected based on changes in the movement signals that are sensed by the inertial sensors of the dental instrument 10. In this embodiment, each gesture event is defined by a single change or a sequence of changes in one or more of the global rotation signals relative to predefined thresholds and/or timing of the changes relative to one another. For example, each gesture event may have a predetermined fluctuation profile based on one or more of the global rotation signals. The fluctuation profile is defined by the magnitude and polarity of the rotation signal against time. As mentioned, some gesture events may be defined by a single-signal fluctuation profile that looks for changes in one signal, while other more complex gestures may be defined by a multi-signal fluctuation profile that looks for a particular sequential or simultaneous signal fluctuation in two or more signals. It will be appreciated that the dental instrument may be configured with any number of single or multi-axis inertial sensors to sense as many inertial signals as is necessary to define a particular gestural movement, depending on design requirements.

Referring to FIG. 19, in this embodiment, gesture events are only detected after the dental instrument is in a steady state. As shown, the gesture detection sub-module firstly looks at step 102 as to whether the dental instrument is being held in a steady state or rest state in space based on the absolute magnitude of the global rotation signals relative to a steady state threshold(s). If a steady state is detected, the gesture detection sub-module 100 then monitors the global rotation signals 99 to discriminate or detect fluctuation profiles that correspond or match to preloaded or predefined gesture event fluctuation profiles. At step 104, if a particular movement or sequence of movements causes a fluctuation in the movement signal or signals that matches a stored fluctuation profile of a gesture event, then the gesture detection sub-module 100 updates the gesture control signal 82 at step 106 with the corresponding gesture control signal representing that detected gesture event, and then returns to step 102 for detecting the next steady state. If no complete gesture event(s) are detected at step 104 after movement of the dental instrument, then the gesture detection sub-module returns to detecting the next steady state at step 102.

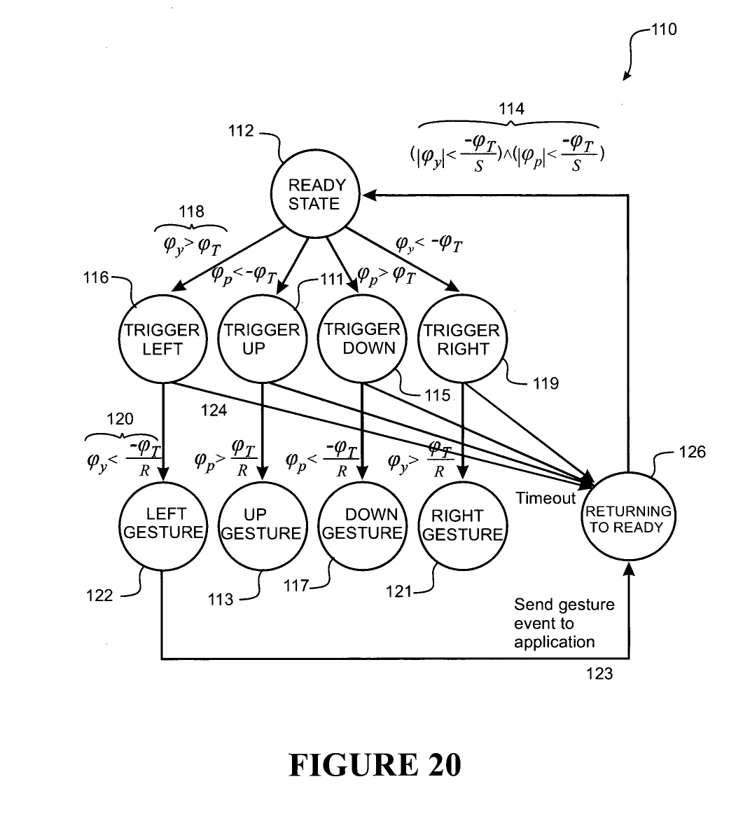

Detection of a gesture event at step 104 in the gesture detection sub-module 100 may be performed for example by a state machine or the like. By way of example only, a state machine 110 is shown in FIG. 20 for detecting simple directional gesture events, such as left gesture, right gesture, up gesture and down gesture. It will be appreciated that the state machine may be modified and expanded to detect other gesture events having different signal(s) fluctuation profiles depending on which gestures are to detected for interaction with the control GUI in the application program.

In this example steady state machine 110, the first step in the state machine 110 is the detection of a steady state 112. In this embodiment, the steady state is defined by the magnitude of the global yaw signal φ_{y} and global pitch signal φₚ being below a steady state threshold as determined by the following equation:

$$( \vert {ϕ}_{y} \vert < \frac{{ϕ}_{T}}{S} ) ⋀ ( \vert {ϕ}_{p} \vert < \frac{{ϕ}_{T}}{S} )$$

where φ_{T} is a trigger threshold and S is scalar such that the steady state threshold is smaller than the trigger threshold. If a steady state is detected, the state machine then monitors the global rotation signals φ_{y}, φₚ for an initial trigger movement causing a change in rotation (angular velocity) in a respective direction of magnitude that exceeds the trigger threshold. The polarity of the trigger threshold defines the direction of movement. For example, in this state machine 110 a positive global yaw signal φ_{y} represents a movement in the left direction relative to the user and a negative global yaw signal φ_{y} represents a movement in the right direction. Likewise, a positive global pitch signal φₚ represents a movement in the downward direction relative to the user and a negative global pitch signal φₚ represents a movement in the upward direction relative to the user. It will be appreciated that the polarity selected may be configured as desired.

In the state machine 110, the horizontal directional gesture events (e.g. left gesture and right gesture) are defined by a fluctuation profile based on the global yaw signal. For example, in this embodiment a horizontal directional gesture event as defined by a movement sequence requiring an initial trigger movement in either the left or right direction followed by a return movement in the opposite direction. By way of example only, a left gesture requires an initial trigger movement in the left direction at 116 such that the global yaw signal exceeds the positive trigger threshold to satisfy the following equation:

φ_{y}>φ_{T}

To complete the left gesture, a return movement in the right direction must then be detected by the global yaw signal exceeding a return threshold having an opposite polarity to satisfy the following equation:

$${ϕ}_{y} < \frac{- {ϕ}_{T}}{R}$$

where R is a scalar such that the return threshold is of at lesser magnitude than the initial trigger threshold such that the return movement need not be of the same magnitude in angular velocity as the initial trigger movement. If a return movement is detected within a predefined timeout period after the initial trigger movement based on the fluctuation of the global yaw signal, then a left gesture is completed and detected at 122 and the corresponding gesture control signal is updated at 123 to send that detected gesture event to the application program for responding accordingly with a corresponding reaction from the control GUI displayed on the host device. If a return movement is not detected within the predetermined timeout to complete the left gesture fluctuation profile, then the state machine exits at 124 to the start state 126 to wait for the next steady state for detecting the next partial or complete gesture event. It will be appreciated that the detection of right gestures follows a global yaw signal fluctuation profile of an opposite polarity as shown by states 119 and 121. Likewise, the vertical directional gestures (up gesture and down gesture) are defined by fluctuation profiles in the global pitch signal having a similar movement sequence of an initial trigger movement and followed by a return movement as shown in the state machine at 111,113 and 115,117.

It will be appreciated that the steady state threshold, trigger threshold, and return threshold may be modified for any one or more of the gesture events as desired. The thresholds effectively dictate the sensitivity of the system to detecting gestures based on movement of the dental instrument. In this embodiment, the steady state machine monitors fluctuation in the instantaneous angular velocity of the rotational movements in various directions, but it will be appreciated that some gestures may be defined by the fluctuation in the absolute orientation or rotational angles of the dental instrument relative to a reference frame in other embodiments.

Alternative Embodiment without Sensing Gestures with Respect to Gravity

The above embodiment employs a control device (e.g. dental instrument) which has a gyroscope sensor for sensing the rotation of the instrument in one or more instrument planes and an accelerometer sensor for sensing orientation of the control device relative to gravity such that gestures oriented with respect to gravity in a global reference frame can be detected. However, it will be appreciated that in an alternative embodiment the control device need not necessarily comprise an accelerometer sensor and may employ a gyroscope sensor for detecting the rotation of the instrument relative to its local reference plane. In such embodiments, the gesture processing module may be configured such that gestures are detected based on rotational movement of the control device relative to the device's own local reference frame.

For example, the instrument rotation signals for φ₁ and φ₂ from the gyroscope could be analysed directly to detect predefined gesture events without orientating the signals relative to gravity or some other global reference frame (i.e. without the movement processing sub-module stage). In this context, the one or more instrument reference planes within which the gyroscope measures rotation of the control device may be aligned with certain parts, marking or axes of the control device. For example, if the control device is a dental instrument having a mirror tool part, the face of the mirror may represent a reference part and the gyroscope may measure rotation in the plane of the face of the mirror and perpendicular to the face of the mirror. The user may then make desired gestural movements of the dental instrument to control the GUI of the dental charting program with knowledge of the orientation of the local reference frame based on position of the mirror face. In such embodiments, the gesturial movements are independent of gravity. They are based on relative movements of the control device itself, regardless of its orientation with respect to gravity.

Alternative Embodiments Using Other Movement Sensing Technologies

The embodiments described above employ onboard inertial sensors for sensing movement of the control device (dental instrument) in space. It will be appreciated that in alternative embodiments the dental charting system may employ any other suitable movement sensing systems or technologies, whether onboard, external, or a combination. By way of example, other suitable movement sensing systems may sense and track 3D position and/or orientation of the control device in space using transmitter-receiver based tracking systems for generating representative movement signals, and may employ RFID tracking technology, ultrasound or RF tracking technology, GPS tracking technology, optical or machine vision tracking, or the like.

Action Processing Module

Reverting to FIG. 15, the action processing module 84 will now be described in further detail. The action processing module is configured to receive and process the switch signals 71,73 from the physical and/or virtual switches of the dental instrument to detect the occurrence of action events from a set of stored predetermined action events and which generates representative action control signals 86 for the application program 12. The dental instrument may be provided with one or more switches and each action event may be defined by switch signals generated by activation and/or deactivation of the one or more switches. An action event may be triggered by single activation of a switch or a sequence of activation of multiple switches or the simultaneous activation of two or more switches. The action processing module monitors the switch signals 71,73 to detect action events. The action events may represent any typical type of GUI control event, including selection, de-selection, escaping, shortcut to menu or any other useful action event that can be used to interact with the control GUI in the application program. For example, one switch may be defined as a main selection switch which is operable to activate icons and the control GUI as will be explained in further detail later. The action event may define short presses or long presses of the button and these action events may different reactions in the control GUI. Another switch may be provided for a shortcut to the home menu in the control GUI or for returning to the previous menu. It will be appreciated that one or more switches may cause any desired reaction in the control GUI depending on the configuration of the action processing module and the application program.

4. Application Program and Control GUI

As previously explained, the user may interact with the control GUI of the application program via gestural movements of the dental instrument and optionally also operation of one or more switches, buttons, control knobs or the like on the dental instrument.

Referring to FIG. 15, the gesture processing module 80 and action processing module 84 of the device driver 24 processes the movement signals sensed by the inertial sensors of the dental instrument and the switch signals generated by any real or virtual switches of the instrument to generate gesture control signal 82 representing detected gesture events and action control signals 86 representing detected action events. The application program 12 is configured with a GUI control engine 90 that is configured to receive and process the control signals 82,86 to cause a corresponding desired and intuitive reaction in the displayed control GUI 94 and application workflow engine 92.

The dental charting system may operate with a standard or conventional dental charting application program which is adapted or modified to include a GUI control engine 90 and control GUI 94 or alternatively a customised dental charting application program comprising a control GUI. In this embodiment, the control GUI is in the form of an icon based GUI, but other formats or types of GUI could alternatively be used such as, but not limited to, tab and drop-down menu based GUIs. The control GUI may have a series of GUI displays, each GUI display comprising an arrangement or configuration of navigatable icons. The icons may represent any desired feature for interacting with the application program including, but not limited to, menus, virtual buttons, data, or actions.

In this embodiment, the display GUIs comprise icons in a fish-eye or carousel traversable format as is known in computer GUIs. It will be appreciated that any other form of icon navigation or display may alternatively be used. By way of example, the display GUIs may have a set of icons that are fixed in location on the screen relative to each other and the user may traverse the icons to highlight or select a desired icon, like in the fish-eye format. Alternatively, the display GUIs may have an arrangement of icons that are movable relative to each other in a controlled manner such that the user can rotate or cycle through the icons to move the desired icon of interest into a highlighted or selected position, like in the carousel format. The gestural movements of the user's hand with the dental instrument causes a corresponding intuitive traversal of the icons (e.g. fish-eye format) or movement of the icons (e.g. carousel format) to enable selection of the icon of interest for further interaction.

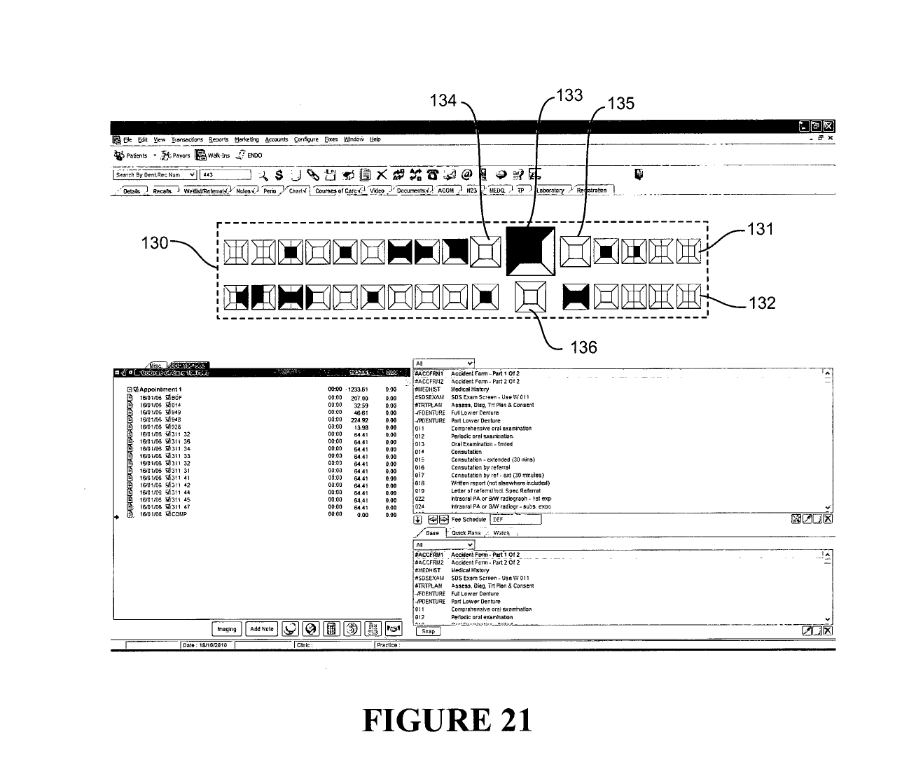

Referring to FIG. 21, a screenshot of one embodiment of a dental charting application program is shown. The display GUI is shown at 130 and represents a dental chart. This display GUI 130 provides two rows of icons, each icon representing one of the patient's teeth. The icons are spilt into an upper row 131 and a lower row 132. The currently selected or highlighted tooth is shown at 133 in a conventional fish-eye format with the selected icon 123 being enlarged or highlighted relative to the remaining icons to signify its current selection. In use, the display GUI 130 may be configured to enable the user to traverse to the desired tooth via corresponding gestural directional movements of the dental instrument 10. For example, from the currently selected icon 133, a left gesture would traverse to selecting the next left adjacent tooth 134 or a right gesture will traverse to the right adjacent tooth 135. Similarly, a downward gesture would traverse to the adjacent lower most tooth 136. Therefore, via a series or sequence of directional gestures (left, right, up, down) with the hand of the user holding the dental instrument, the user can select the desired tooth. Each of the tooth icons shows the various surfaces of the tooth and is colour coded to represent the patient data showing the condition and/or treatment of the various surfaces of each tooth.

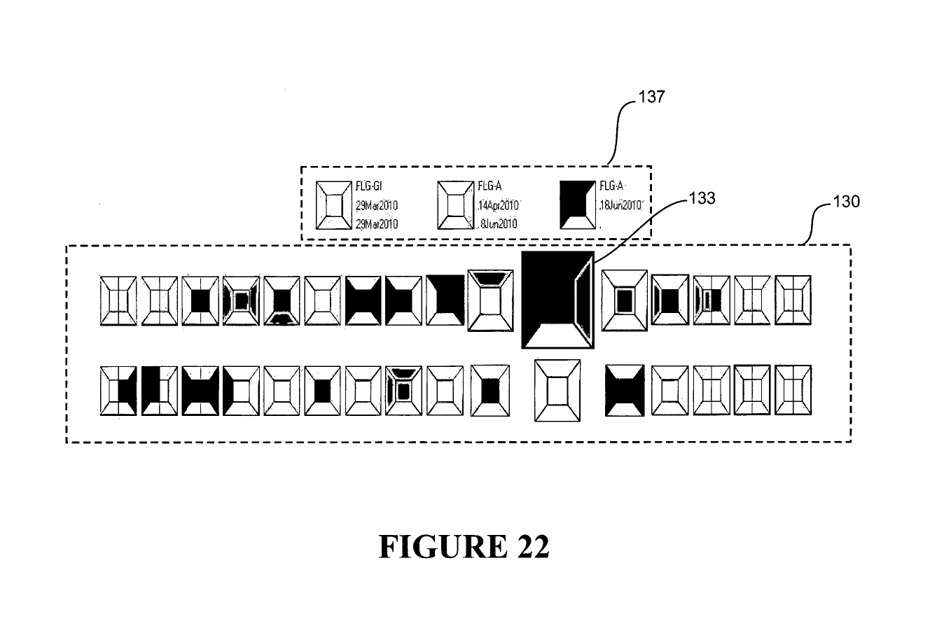

Referring to FIG. 22, the display GUI may be configured to display a progress or history sub-chart 137 for each individual tooth as it is selected. In this screenshot, 137 represents the sub-chart history of treatment and/or condition for the selected tooth 133. As the user traverses to a different tooth a new tooth history sub-chart will be displayed in 137.

Depending on the workflow of the application, the selected tooth 133 may be activated (like a button) by a user to present a new display GUI having one or more icons for traversing and/or activating to progress to the next stage in the application program. The GUI control engine 90 may be configured such that activation of the icon may be triggered by a particular gesture event, or alternatively an action event caused by activation of one or more physical or virtual switches provided on the dental instrument.

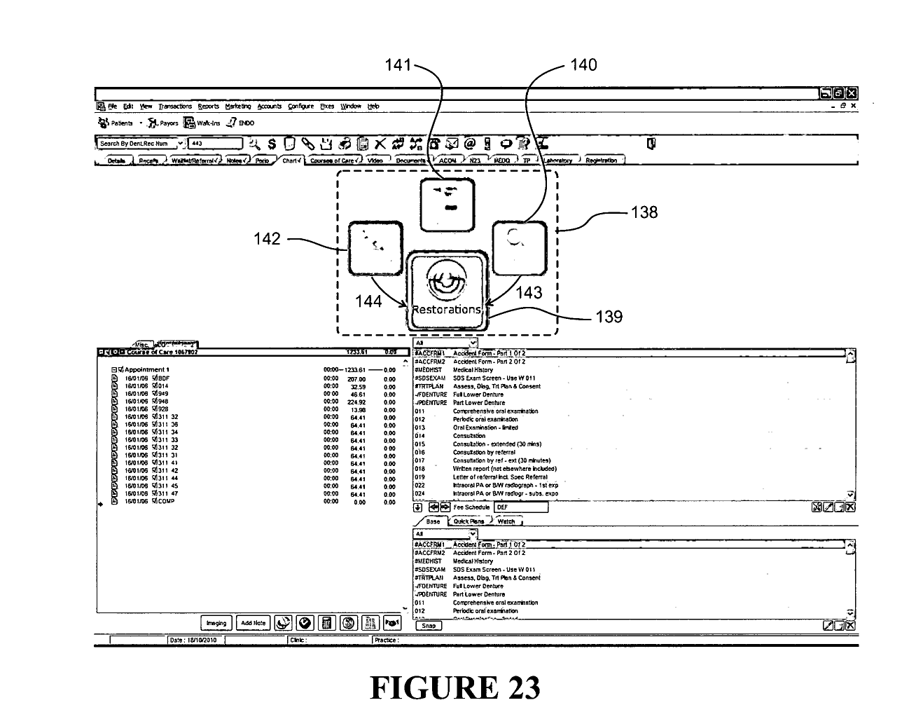

By way of example, FIG. 23 shows another type of display GUI 138 comprising icons in a rotatable carousel format. In this format, the icons 139-142 are configured to rotate clockwise or anticlockwise based on the gestural movements of the dental instrument. The selected or highlighted icon for activation is the icon displayed at the lower most position in the carousel as shown at 139. By way of example, a left gesture movement event may cause a corresponding clockwise rotation of the carousel so as to move icon 140 into the lower most selected position as shown by arrow 143 and a right gesture may cause an anticlockwise rotation moving icon 142 into the selected lower most position as shown by arrow 144. In an alternative embodiment, the display GUI 138 need not necessarily be in a carousel rotating format and may have stationary icons which may be traversed for selection via directional gestural movements as with the dental chart display GUI 130. Again, the selected icon 139 may be activated by a gestural movement or alternatively operation of one or more activation switches on the dental instrument to move to the next stage in the application workflow. For example, in one embodiment activation of the selected icon may be achieved by triggering of a virtual switch via the tap detection module as previously explained. For example, a single tap may advance the workflow application to the next stage while a double tap may revert back to the previous GUI display or stage or alternatively may jump or escape the program back to the main home display GUI. The programming of the GUI control engine in response to the action events (e.g. switch activations, single taps, double taps, long press, short press) and gesture events (e.g. directional gesture movements, shaking, dropping) may be configured as desired to control the control GUI in different intuitive ways.

Application Workflow

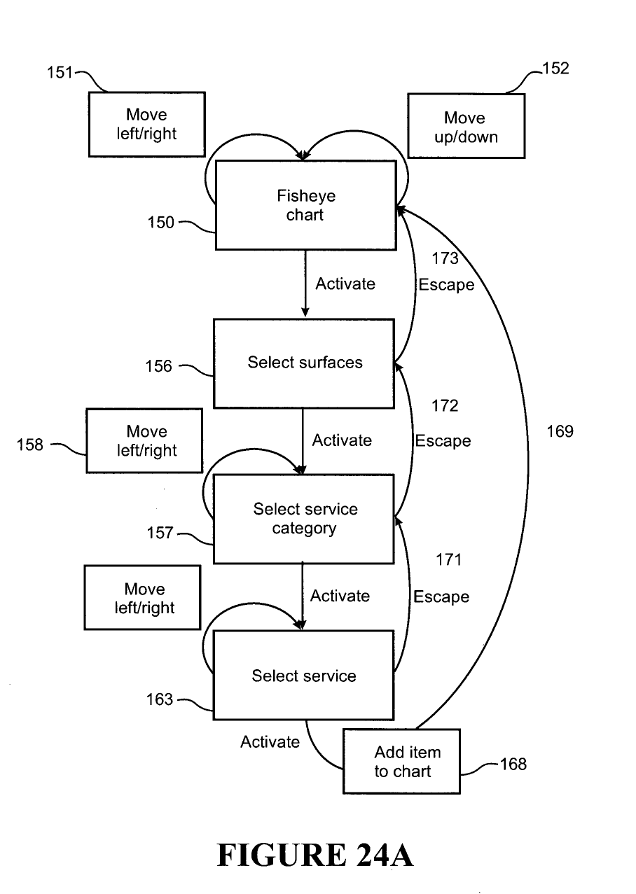

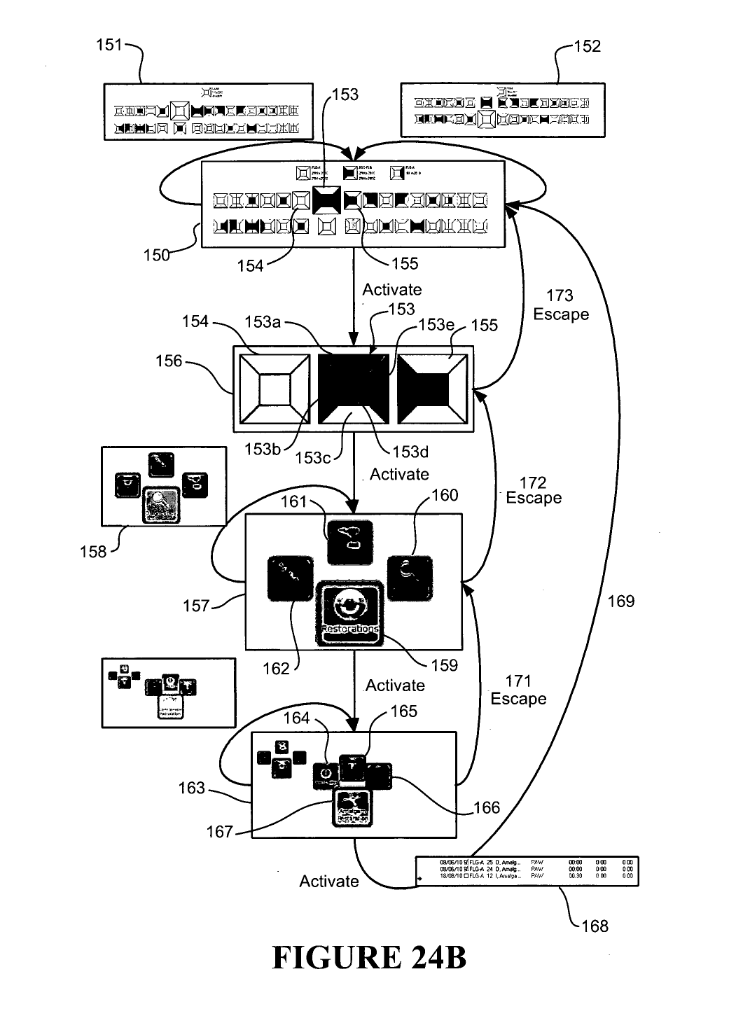

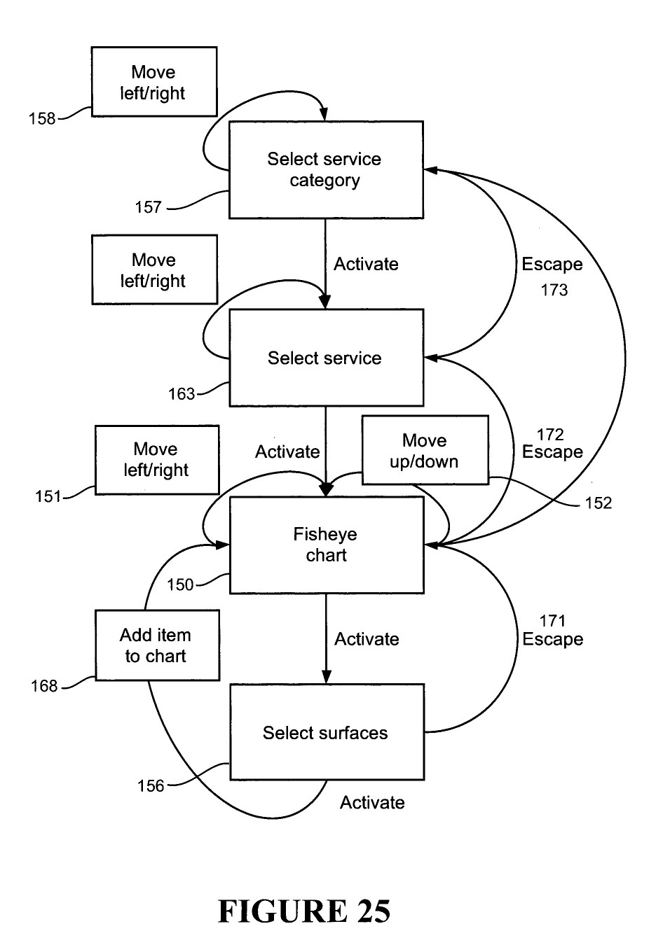

It will be appreciated that the dental charting application program may be configured to respond to detected gesture events and action events by movement and operation of the portable dental instrument in a number of ways depending on the workflow of the application. It will be appreciated that both simple directional gestures and more complex gestures (for example shaking, dropping, twisting or rolling) with the dental instrument may be configured to have an intuitive or corresponding response or reaction in the display GUIs of the application program user interface. By way of example only, a typical first type of application workflow or module of a dental charting application program will be described with reference to FIGS. 24A-25.

FIGS. 24A and 24B show an example of a first workflow in the dental charting application program for base charting, which is for recording historical or existing treatment of patients' teeth. For example, once the dentist has examined the patient and decided on the treatment that is going to be performed or the condition of the tooth for updating in the records, they may then use base charting workflow application to record the treatment or condition against the relevant tooth. FIG. 24A shows the workflow at a block diagram level with FIG. 24B showing an example of the corresponding display GUIs that the user is presented with on the display of the PC.

Once this first base charting workflow is initiated in the dental charting application program, the user is presented with a first dental chart display GUI at step 150. The first display GUI may be in the form of a fish-eye dental chart 136 and history sub-chart 137 as explained with reference to FIGS. 21 and 22. At the first step 150, the dentist or operator may use directional gestural movements while holding the dental instrument as previously explained to traverse the icons to select the desired tooth. For example, they may move left and right along the upper or lower rows of teeth at 151 via left and right hand gestures or alternatively up and down between the upper and lower teeth using up and down hand gestures as shown at 152. Once the desired tooth is highlighted or selected, the user may activate that icon by triggering a particular action switch on the instrument or alternatively via a gestural movement such as a single tap, or double tap, or some other simple or more complex gestural movement.

Once the selected tooth icon 153 is activated, the workflow moves to the second step 156 and presents a new second display GUI showing an enlarged selected tooth icon 153 and having as a reference frame the adjacent teeth icons 154,155 displayed on either side. In this second step 156, the display GUI is configured to enable the user to traverse the surfaces 153a-153e of the tooth with gestural movements, such as up, down, side-to-side to highlight the surface or surfaces that require treatment or updating in the records. Once the desired surface is highlighted, it may be activated by causing an activation event via operation of a particular action switch (e.g. selection switch) on the dental instrument or via a particular gestural movement, such as a single tap or double tap or any other gestural movement. Once all of the desired surfaces are selected, the user may trigger an activation event to move to the third step 157. For example, the surface 153a of tooth 153 has been selected as shown in FIG. 24B. In one form, each tooth surface may be selected one at a time by highlighting and then activating of a selection switch, before then moving to the third step 157. In an alternative form, multiple tooth surfaces may be selected at one time by holding a selection switch and gesturing to highlight the desired multiple tooth surfaces, i.e. a click and drag configuration to selection, before moving to the third step 157.

In the third step 157 of the workflow, the user is presented with a service category display GUI of the type 138 described with reference to FIG. 23, i.e. carousel arrangement of icons as shown at 158 and previously explained. The user may use left gestures and right gestures to rotate the carousel to select the desired service category. In this case, four service category icons are presented: restorations 159, diagnostics 160, oral surgery 161, and prevention 162. The carousel is rotated to select the desired service category for applying to the selected surface 153a of the selected tooth 153. Once the desired service category is selected (in this case restorations 159) the user operates the activation switch or carries out an activation gesture to cause an action event to move to step 163.

At the fourth step 163, the user is presented with a fourth display GUI representing the services under restorations. By way of example, the service display GUI may also be in the form of a rotatable carousel format. Again, the user may traverse the service icons 164-167 and may activate the desired service icon to cause the workflow to move to the final step 168 of the workflow application where the selected service is recorded against the selected surface(s) of the selected tooth, along with the date any other relevant information, against the electronic patient record. In other words the service is added to the dental chart on the selected surface(s) of the selected tooth. The workflow application then returns to the initial first fish-eye dental chart as shown by arrow 169.

As shown, the user may backtrack to previous display menus by triggering an escape or return event via gestural movements or operation of one or more of the escape switches on the dental instrument as shown at 171-173. In addition, the workflow may be provided with a shortcut for traversing back to the start GUI or home menu and the shortcut may be triggered in response to a particular corresponding gesture event (via gestural movement) or activation of an action event (via operation of the switches).

FIG. 25 shows an alternative base charting workflow in which the service category 157 and service 163 are selected prior to teeth and teeth surfaces. Like reference numbers represent like steps. In this embodiment, once the service 163 is selected, the service may be applied to one or multiple teeth with the presented dental chart at step 150. For example, once a tooth is selected at step 150, the workflow moves to the surface selection at step 156. Once the surfaces are selected, the activation event is triggered to register the service against the surfaces of the selected tooth and the workflow then returns automatically into the dental chart at step 154 for selection of further teeth if desired. Again, an escape event may be triggered via a desired gestural movement or escape switch operation to traverse back to a previous step or menu and a master escape event 174 may be provided for jumping back to the first step of selecting the service category if desired.

It will be appreciated that various other workflows may be implemented in the dental charting application program that are configured to be controlled by user interaction with a control GUI of the type explained above. Further, the dental charting application program may have additional workflows or functionalities that are controlled by more conventional user interface format via conventional computer inputs such as keyboard and mouse. For example, application program may have workflows that are controlled by conventional computer inputs, such as mouse and keyboard, via GUIs with tab and drop-down menus, and workflows that are configured to be controlled by a control GUI that interacts with gestural movements of the dental instrument. For example, the workflows in which the dental instrument controls the interaction with the program may be initiated by selecting the workflow from a conventional tab or Windows based user interface via a mouse and/or keyboard. Once the dental instrument controlled workflow is initiated, interaction and control with the GUI is provided by gestural movements and/or switch operation of the dental instrument, and optionally the conventional computing inputs such as keyboard and/or mouse may provide overrides if needed. Alternatively, the dental charting application may be configured such that all aspects are fully controlled by the dental instrument via gestural movement and/or switch operation.

The foregoing description of the invention includes preferred forms thereof. Modifications may be made thereto without departing from the scope of the invention as defined by the accompanying claims.

1. A dental charting system, comprising:
a control device configured to be held by a user or which is mounted to another device held by a user and the control device comprising one or more movement sensors for sensing movement of the control device in space and generating representative movement signals;
a gesture processing module configured to receive and process the movement signals to detect the occurrence of a gesture event from a set of predetermined gesture events, and which generates representative gesture control signals; and
a dental charting application program running on a host device and which provides a graphic user interface on a display associated with the host device for interacting with the application program, the application program being configured to receive and process the gesture control signals to enable interaction with the graphic user interface by the user via hand gesture movements of the control device.

2. (canceled)

3. A dental charting system according to claim 1 wherein the control device is a dental instrument, or is configured to be fixedly mounted or releasably mounted to a dental instrument.

4-11. (canceled)

12. A dental charting system according to claim 1 wherein the control device has a predefined reference axis, and wherein the control device is substantially elongate and the reference axis is aligned or parallel with the longitudinal axis of the control device.

13. (canceled)

14. A dental charting system according to claim 12 wherein the movement sensor(s) are inertial sensors and generate movement signals that represent movement of the control device with respect to a local reference frame of the control device, and wherein the inertial sensor(s) comprise a gyroscope sensor mounted to or within the control device that is configured to sense rotation of the reference axis of the control device with reference to one or more control device reference planes defined by the local reference frame and generate representative control device rotation signals.

15. (canceled)

16. A dental charting system according to claim 12 wherein the movement sensor(s) generate movement signals that represent rotation of the reference axis with reference to one or more global reference planes oriented with respect to gravity, and wherein the global reference planes comprise either or both of the following: a horizontal plane with reference to gravity, and a vertical plane with reference to gravity, and wherein the global reference planes are aligned with the reference axis of the control device.

17-18. (canceled)

19. A dental charting system according to claim 12 wherein the movement sensors comprise: an accelerometer sensor mounted to or within the control device that is configured to sense the orientation of the reference axis of the control device with respect to gravity and generate representative orientation signals; and a gyroscope sensor mounted to or within the control device that is configured to sense rotation of the reference axis of the control device with reference to one or more control device reference planes and generate representative control device rotation signals.

20. A dental charting system according to claim 19 wherein the accelerometer sensor is a three-axis accelerometer which is configured such that at least one of the sensor axes is co-aligned or parallel with the reference axis of the control device, and wherein the gyroscope sensor is a two-axis gyroscope that is configured to sense rotation of the reference axis with reference to two perpendicular control device reference planes, and wherein either or both of the control device reference planes have an orientation that is co-aligned or parallel to the reference axis of the control device.

21-23. (canceled)

24. A dental charting system according to claim 19 wherein the gesture processing module comprises a movement processing sub-module and a gesture detection sub-module, and wherein the movement processing sub-module is configured to receive and process the accelerometer and gyroscope signals to generate global rotation signals representing the rotation of the reference axis of the control device with reference to one or more global reference planes oriented with respect to gravity, and wherein the gesture detection sub-module is configured to receive the global rotation signals from the movement processing sub-module, process the global rotation signals to detect the occurrence of gesture events, and generate representative gesture control signals representing any detected gesture events for sending to the host device, and wherein the gesture detection sub-module comprises a set of predetermined gesture events for detecting, each gesture event in the set being defined by a movement sequence based on the change of one or more of the global rotation signals relative to one or more predefined signal threshold(s).

25. (canceled)

26. A dental charting system according to claim 24 wherein the movement processing sub-module is configured to process the accelerometer orientation signals to extract the pitch and roll of the reference axis of the control device with respect to gravity and generate representative pitch and roll signals; and is further configured to convert the control device rotation signals from the gyroscope into global rotation signals representing the rotation of the reference axis of the control device with respect to the one or more global reference planes based on the pitch and roll signals, and wherein the global rotation signals comprise a global yaw signal representing the rotation of the reference axis of the control device in the horizontal plane with respect to gravity and a global pitch signal representing the rotation of the reference axis in the vertical plane with respect to gravity.

27-30. (canceled)

31. A dental charting system according to claim 24 wherein each gesture event is defined by a signal fluctuation profile in regard to one or more of the global rotation signals, and wherein each signal fluctuation profile is defined by signal magnitude and polarity against time.

32. (canceled)

33. A dental charting system according to claim 24 wherein the set of predetermined gesture events comprises one or more directional gesture events selected from: left gesture, right gesture, up gesture, and down gesture, and wherein each of the directional gestures is defined by a movement sequence comprising an initial trigger movement causing a change in rotation in a respective direction of a magnitude that exceeds a trigger threshold, followed by a return movement causing a subsequent change in rotation in the opposite direction of a magnitude exceeding a return threshold, and wherein the movement sequence requires the return movement to occur within a predefined timeout period after detection of the initial trigger movement to complete the movement sequence such that a direction or gesture event is registered as occurring.

34-39. (canceled)

40. A dental charting system according to claim 1 wherein the control device further comprises one or more control switches that are operable by the user to generate switch signals representing operation of the one or more switches, and wherein the switch signals are processed into action control signals that are received by the application program to enable further interaction with the application program by the user, and wherein the action control signals represent the occurrence of action events from a set of predetermined action events that enable the user to interact with a GUI of the application program via operation of the switches in combination with navigating the GUI via gesture control signals generated by user hand gestural movements of the control device.

41-42. (canceled)

43. A dental charting system according to claim 40 wherein the control switches comprise one or more virtual switches that are activatable via movement of the control device as detected by one or more of the movement sensors, and wherein at least one of the virtual switches is a tap detection module associated with the movement sensors that is configured to detect taps of the control device and generate representative switch signals on detection of a tap or tap sequence.

44-45. (canceled)

46. A dental charting system according to claim 1 wherein the control device is a portable handheld dental instrument comprising an elongate handle portion and a tool portion extending from an end of the handle portion, and the handle portion comprising a substantially elongate main body within which the movement sensors are mounted and an outer casing that is configured to substantially surround at least a substantial portion of the main body.

47. A dental charting system according to claim 46 wherein the outer casing of the dental instrument is in the form of an elongate sleeve that fits over at least a substantial portion of the main body.

48. A dental charting system according to claim 46 wherein the dental instrument is provided with one or more touch switches mounted to the surface of the main body and wherein the main body and outer casing are configured such that there is an air gap between the touch switches and the inner surface of the outer casing in the vicinity of the touch switches.

49-50. (canceled)

51. A dental charting system according to claim 48 wherein the outer casing of the dental instrument is resiliently deformable in a region aligned with the touch switches of the main body.

52. A dental charting system according to claim 46 wherein the tool portion is a dental mirror that is releasably mounted to the handle portion of the dental instrument.

53. A dental charting system according to claim 1 wherein the GUI of the application program comprises an arrangement of selectable icons that are traversable by a user via gestural movements of the control device in space.

54-55. (canceled)

56. A dental charting system, comprising:
a control device configured to be held by a user or which is mounted to another device held by a user;
a movement sensing system configured to sense movement of the control device in space and generating representative movement signals;
a gesture processing module configured to receive and process the movement signals to detect the occurrence of a gesture event from a set of predetermined gesture events, and which generates representative gesture control signals; and
a dental charting application program running on a host device and which provides a graphic user interface on a display associated with the host device for interacting with the application program, the application program being configured to receive and process the gesture control signals to enable interaction with the graphic user interface by the user via hand gesture movements of the control device.

57-63. (canceled)
